(19) United States
(12) Patent Application Publication
(10) Pub. No.: US 2022/0079437 A1
(43) Pub. Date: Mar. 17, 2022

(54) MEASUREMENT DEVICE AND METHOD FOR INTERNAL PRESSURE OR RIGIDITY OF MEASURED OBJECT

(71) Applicant: TONESY INC., Machida-shi, Tokyo (JP)

(72) Inventors: Kosei SUZUKI, Chosei-gun, Chiba (JP); Hidenaga KOBASHI, Machida-shi, Tokyo (JP)

(73) Assignee: TONESY INC., Machida-shi, Tokyo (JP)

(21) Appl. No.: 17/416,842

(22) PCT Filed: Dec. 23, 2019
(86) PCT No.: PCT/JP2019/050299

§ 371 Date: Jun. 21, 2021

(30) Foreign Application Priority Data
Dec. 21, 2018 (JP) 2018-239414

(51) Int. Cl. A61B 3/16

(52) U.S. Cl. CPC A61B 3/16; A61B 2562/0247

(57) ABSTRACT

A device and a method for measuring internal pressure of a measured object are disclosed. A measurement device includes a pressing portion and a measurement portion, and measures the internal pressure of the measured object. The pressing portion has, at a tip, a contact surface that comes into contact with the surface of the measured object. The measurement portion includes a first sensing means that continuously senses repulsive force from the measured object when the contact surface is brought into contact with the surface of the measured object and the pressing portion is pressed toward the measured object, and a second sensing means that continuously senses, when the contact surface is brought into contact with the surface of the measured object and the pressing portion is pressed toward the measured object: speed, acceleration or distance of movement of the pressing portion in a direction of the measured object.

### CROSS-REFERENCE TO RELATED APPLICATIONS

This application is a U.S. National Stage application of International Patent Application No. PCT/JP2019/050299 filed on Dec. 23, 2019, which claims priority to Japanese Patent Application No. 2018-239414 filed on Dec. 21, 2018, the entire contents of both of which are incorporated herein by reference in their entireties for all purposes.

### TECHNICAL FIELD

The present disclosure relates to a measurement device and a measurement method for internal pressure of a measured object.

### BACKGROUND ART

Various proposals have been made conventionally to measure internal pressure of a measured object based on the amount of deformation and load when a measurement instrument is brought into contact with the measured object and the measured object is pressed.

For example, to measure intraocular pressure, an applanation tonometer and an indentation tonometer in which a probe is brought into contact with the cornea have been used for a long time. Various proposals have been made regarding the measurement of intraocular pressure.

In Patent Literatures 1 to 7, various proposals are made regarding the measurement of intraocular pressure through the eyelid.

Patent Literature 2 proposes a tonometer including a pressurizing body, a driving means, a load sensor, and a calculation means. The pressurizing body presses the eye to be measured through the eyelid. The driving means generates a pressing force of the pressurizing body. The load sensor senses the amount of load applied to the pressurizing body. The calculation means obtains intraocular pressure based on the amount of load sensed by the load sensor with respect to the amount of displacement of the eyeball generated by the pressing of the pressurizing body. Pressing is performed by moving the pressurizing body at a constant speed, and at that time, the calculation means measures intraocular pressure based on the time course of the load detected by the load sensor.

Patent Literatures 3 and 4 propose methods and devices for measuring intraocular pressure through a closed eyelid.

Patent Literatures 8 to 12 propose a method to measure the force applied to a structure from the outside by placing a pressure sensor inside a sealed structure or a structure that has weak ventilation by measuring the internal pressure of the structure. Patent Literatures 13 to 15 propose a method of measuring the depth of chest compressions performed during cardiopulmonary resuscitation using an accelerometer.

### CITATION LIST

### Patent Literature

Patent Literature 1: JP H6-38930 A
Patent Literature 2: JP H6-105811 A
Patent Literature 3: JP H8-280630 A
Patent Literature 4: JP 2002-501801 A
Patent Literature 5: US 2004/0267108 A1
Patent Literature 6: US 2010/0152565 A1
Patent Literature 7: U.S. Pat. No. 6,440,070
Patent Literature 8: US 2018/0284936 A1
Patent Literature 9: US 2011/0007023 A1
Patent Literature 10: US 2009/0174687 A1
Patent Literature 11: US 2010/0103137 A1
Patent Literature 12: US 2014/0069212 A1
Patent Literature 13: U.S. Pat. No. 6,306,107
Patent Literature 14: U.S. Pat. No. 7,220,235
Patent Literature 15: JP 4689979 B2

### SUMMARY OF INVENTION

### Technical Problem

An object of the present invention is to propose a measurement device and a method for the internal pressure of a measured object. The device is very small and easy to use for anyone.

### Solution to Problem

### Advantageous Effects

According to the present disclosure, it is possible to provide a measurement device and a method for internal pressure of a measured object. The device is very small and easy to use for anyone.

### BRIEF DESCRIPTION OF DRAWINGS

FIG. 1 illustrates a cross-section view of an example of a measurement device according to an embodiment of the present invention, where a part is omitted.

FIGS. 2(a)-2(c) illustrate another example of the measurement device according to an embodiment of the present invention. FIG. 2(a) is a cross section view where a part is omitted. FIG. 2(b) is a plan view. FIG. 2(c) is a side view.

FIG. 3 illustrates a cross-section view of another example of the measurement device according to an embodiment of the present invention, where a part is omitted.

FIG. 4 illustrates a cross-section view of another example of the measurement device according to an embodiment of the present invention, where a part is omitted.

FIG. 5 illustrates a cross-section view of another example of the measurement device according to an embodiment of the present invention, where a part is omitted.

FIG. 6 illustrates a cross-section view of another example of the measurement device according to an embodiment of the present invention, where a part is omitted.

FIG. 7 illustrates a cross-section view of another example of the measurement device according to an embodiment of the present invention, where a part is omitted.

FIG. 8 illustrates a cross-section view of another example of the measurement device according to an embodiment of the present invention, where a part is omitted.

FIGS. 9(a) and 9(d) illustrate the measurement principle of the measurement device according to an embodiment of the present invention. FIG. 9(a) illustrates the relationship between a lapse of time from the start of measurement (horizontal axis) and the output (acceleration) (vertical axis) from the second sensing means provided in the measurement device. FIG. 9(b) illustrates the relationship between a lapse of time from the start of measurement (horizontal axis) and the speed of movement of the pressing portion of the measurement device (vertical axis). FIG. 9(c) illustrates the relationship between a lapse of time from the start of measurement (horizontal axis) and the distance of movement of the measurement device toward the measured object (vertical axis). FIG. 9(d) illustrates the relationship between a lapse of time from the start of measurement (horizontal axis) and the repulsive force F.

FIGS. 10(a) and 10(b) illustrate the measurement principle of the measurement device according to an embodiment of the present invention. FIG. 10(a) illustrates an example of the relationship between D and F, where D is the distance that the pressing portion of the measurement device moves in the direction of the measured object, and F is the strength of the repulsive force from the measured object with respect to the pressing by the pressing portion. In the Figure, the curve represented by the upper solid line and the broken line below have the same spring constants (ΔF/ΔD) on the right side of the graph where ΔF/ΔD is stable, and the curve represented by the solid line further below has a different spring constant from the two lines above. FIG. 10(b) illustrates an example of the relationship between D and ΔF/ΔD. In the Figure, the curves represented by the upper solid line and the broken line below have the same spring constants (ΔF/ΔD) on the right side of the graph where ΔF/ΔD is stable, and the curve represented by the solid line further below has a different spring constant from the two lines above.

FIGS. 11(a)-11(c) illustrate the measurement principle of the measurement device according to an embodiment of the present invention. FIG. 11(a) illustrates the relationship between the instantaneous spring constant ΔF/ΔD obtained by the measurement device according to an embodiment of the present invention and the measurement result of intraocular pressure measurement using a conventionally known device/method, where data from multiple human subjects are plotted and a method of deriving a transformation curve is explained. FIGS. 11(b) and (c) show methods of calibration with calibration curves using data from one subject.

FIG. 12 illustrates the measurement principle of the measurement device according to an embodiment of the present invention, showing an example of the relationship between the strength of the repulsive force F from the measured object with respect to the pressing portion of the measurement device and the output of the first sensing means.

FIGS. 13(a) and 13(b) illustrate cross-section views of another example of the measurement device according to an embodiment of the present invention, where a part is omitted.

FIG. 14 illustrates a cross-section view of another example of the measurement device according to an embodiment of the present invention, where a part is omitted.

### DESCRIPTION OF EMBODIMENTS

The measurement device of this example measures the internal pressure of the measured object.

The measurement device of this example includes a pressing portion and a measurement portion.

The pressing portion is provided at a tip with a contact surface that comes into contact with the surface of the measured object.

The measurement portion includes a first sensing means and a second sensing means.

The pressing portion and the measurement portion can be in a form in which the pressing portion is supported by the measurement portion to be movable so that the pressing portion can move from the tip toward the pressing portion rear, which is the opposite side of the pressing portion from the tip.

The pressing portion rear can be in a form that is supported by the measurement portion. Various forms can be exemplified as the form in which the pressing portion rear is supported by the measurement portion, and for example, the following form can be exemplified. (1) A structure in which the pressing portion and the measurement portion are combined. For example, a structure in which the pressing portion is fixed to the measurement portion on the pressing portion rear and the pressing portion is erected on the measurement portion. (2) A structure in which the pressing portion and the measurement portion are separate, and the pressing portion rear can be attached to/removed from the measurement portion.

The first sensing means continuously senses repulsive force from the measured object when the contact surface is brought into contact with the surface of the measured object and the pressing portion is pressed toward the measured object.

As described above, the second sensing means continuously senses, when the contact surface is brought into contact with the surface of the measured object and the pressing portion is pressed toward the measured object, any one of

speed of movement at which the pressing portion moves in a direction of the measured object,

acceleration of the pressing portion in the movement in the direction of the measured object, or

distance of movement of the pressing portion in the direction of the measured object.

The measurement by the measurement device of this embodiment is performed, for example, as follows.

By using the measurement device described above, when the contact surface is brought into contact with the surface of the measured object and the pressing portion is pressed toward the measured object, repulsive force F from the measured object and any one of: speed of movement at which the pressing portion moves in the direction of the measured object, acceleration of the pressing portion in the movement in the direction of the measured object, or distance of movement of the pressing portion in the direction of the measured object are continuously sensed.

A case described in the following is when the distance of movement D of the pressing portion in the direction of the measured object is continuously sensed by the second sensing means, when the contact surface is brought into contact with the surface of the measured object and the pressing portion is pressed toward the measured object.

A sensor that outputs digital values may be used for the first sensing means and the second sensing means. Then, the sensing is discrete, but the discrete sensing can be regarded as continuous sensing by appropriately selecting the output data interval, as described later. Thus, the continuous sensing by the sensing means such as the first sensing means, the second sensing means, and the like in the present invention is a concept including the case where the sensing means outputs digital values.

Hereinafter, in the present specification and drawings, the changes in the repulsive force F and the distance of movement D in a very short time (Δt) will be defined as ΔF and ΔD, respectively.

The repulsive force (F) when the measured object is deformed by a press with the pressing portion increases as the distance of movement (D) of the pressing portion in the direction of the measured object increases. As a conventional method for measuring the internal pressure of a measured object from the outside, a measurement based on the repulsive force when a predetermined amount of deformation is applied to the measured object has been used.

In this embodiment, description will assume that the measured object is composed of a surface film and contents. When the contents can be easily compressed such as gas or the like, a change in the internal pressure changes the pressure applied to the film. When the contents are difficult to be compressed such as water or the like, a change in the volume of the content changes the pressure applied to the film, so the change in the volume of the content can be regarded as a change in the internal pressure. When the pressure applied to the film from inside changes, the rigidity of the film changes. Therefore, if the relationship between the internal pressure and the rigidity of the film is known in advance, the internal pressure can be measured by measuring the rigidity of the film. The rigidity of the surface film of the measured object independent of the pressure received from the contents can be excluded by calibration with a calibration curve, as described later.

A measured object such as an eyeball or the like that is composed of a surface film and contents will be modeled as a spring to measure the internal pressure. In the range where the distance of movement D of the pressing portion in the direction of the measured object is short, the measured object can be modeled as a linear spring. The distance of movement D of the pressing portion in the direction of the measured object necessary to measure the internal pressure is short. When Hooke's law “repulsive force F=spring constant×distance of movement D” (where D is distance of movement of pressing portion toward object) is applied to the instantaneous distance of movement of the pressing portion, equation “ΔF=instantaneous spring constant×ΔD” holds. Then, the instantaneous spring constant (ΔF/ΔD) during the continuous sensing is obtained for each interval of time. The larger the internal pressure, the larger the instantaneous spring constant (ΔF/ΔD).

If the spring constant is stable during Δt, the Δt may be long when obtaining ΔF and ΔD. The actual Δt should be adjusted according to an embodiment. When the sensor used for the first sensing means and the second sensing means is digital output, the minimum possible Δt is determined by the output data interval (interval of discrete data), but an interval of time longer than the output data interval may be used as Δt. When analog output, the minimum possible Δt is determined by the sampling interval of an following ΔD (analog-to-digital) converter in the subsequent stage, but an interval of time longer than the sampling interval may be used as Δt.

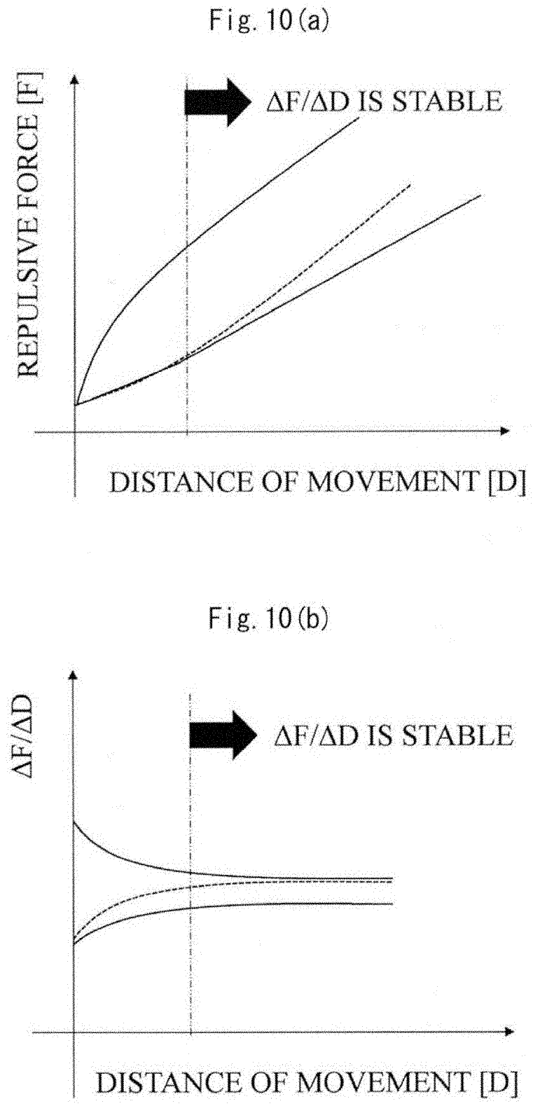

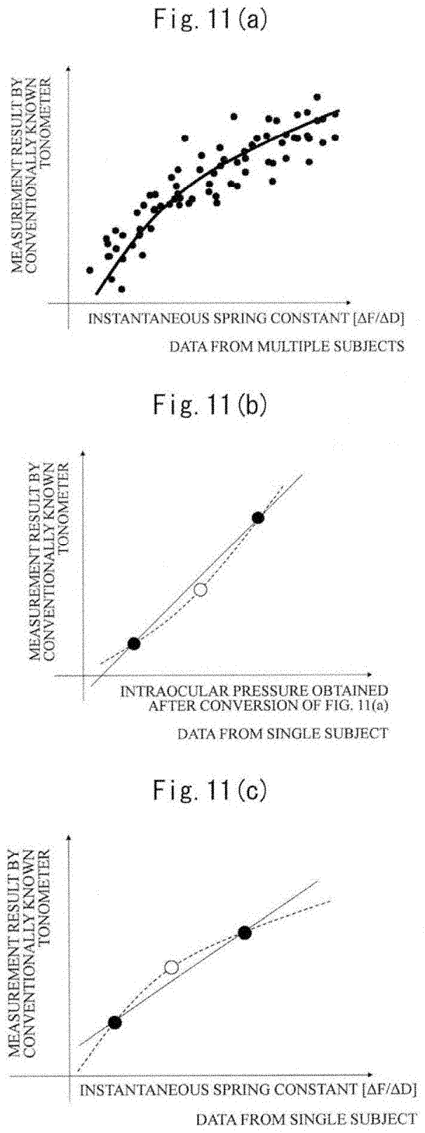

If the relational expression between the actual internal pressure of the measured object obtained by a conventionally known method and the spring constant (ΔF/ΔD) obtained by the measurement device are known in advance, the internal pressure of the measured object can be measured by the measurement device. This will be described using FIGS. 10 and 11.

FIG. 10(a) illustrates an example of the relationship between the repulsive force F from the measured object and the distance of movement D of the pressing portion in the direction of the measured object, when the contact surface is brought into contact with the surface of the measured object and the pressing portion is pressed toward the measured object.

As illustrated in FIGS. 10(a) and 10(b), regarding the repulsive force F and the distance of movement D that are continuously sensed by the first sensing means and the second sensing means respectively, when pressed to some extent, the instantaneous spring constant (ΔF/ΔD) during the continuous sensing becomes stable and the correlation of ΔF/ΔD with the internal pressure becomes high.

The rigidity of the surface film of the measured object independent of the pressure received from the contents is distinguished from the stiffness/thickness of the outermost layer that covers the surface film of the contents from further outside. When measuring intraocular pressure through the eyelid with the measurement device, the stiffness/thickness of the outermost layer that covers the surface film of the contents from further outside is related to the skin-side layer of the eyelid. Since this layer has a small spring constant and is a short spring, it reaches the compression limit keeping the repulsive force very small when pressed. Therefore, its rigidity is regarded as negligible. Hereinafter, this layer will be referred to as “the outermost surface layer whose rigidity is negligible”. The outermost surface layer whose rigidity is negligible affects the repulsive force F at the start of pressing (time t=0 and D=0), which is the reference point. However, since the rigidity of the film is measured as an instantaneous spring constant ΔF/ΔD by the measurement device, the outermost surface layer whose rigidity is negligible does not affect the rigidity of the film.

When intraocular pressure is measured through the eyelid by the measurement device, the rigidity of the film is measured as an instantaneous spring constant ΔF/ΔD, assuming that the following two components are combined.

(1) Rigidity of the eyeball-side layer of the eyelid and the surface of the eyeball that is independent of intraocular pressure.

(2) Rigidity of the surface of the eyeball that changes according to intraocular pressure.

Component (1) generally corresponds to the rigidity of the surface film of the measured object independent of the pressure received from the contents as described above. Components (1) and (2) can be distinguished by creating a calibration curve, as described later. What we want to find here is component (2).

There are various methods for to determine that ΔF/ΔD is stable during the continuous sensing, but any method may be used. For example, ΔF/ΔD may be adopted at the time when the variation in multiple consecutive values of ΔF/ΔD is minimized in the period from the start of pressing to the maximum depth of pressing.

In FIGS. 10(a) and 10(b), the following three factors can be reasons why the instantaneous spring constant ΔF/ΔD is unstable near the start of each press (time t=0 and D=0).

(1) The repulsive force F and ΔF from the outermost layer (the skin-side layer of the eyelid) with negligible rigidity should be very small, but ΔD is also very small at the start of each press. Therefore, the values of ΔF/ΔD become unstable.

(2) Immediately after the start of each press, parameters ΔF and ΔD during Δt are very small, and the signal to noise (S/N) ratios of the sensors used for the first sensing means and the second sensing means are low.

(3) The ideal is to press in stationary position, but if not completely stationary, values ΔF and ΔD immediately after the start of each press do not reflect the pressing.

As illustrated in FIGS. 10(a) and 10(b), when pressing until the above three factors become negligible, the instantaneous spring constant ΔF/ΔD during continuous sensing stabilizes and has a high correlation with the internal pressure of the measured object. Hereinafter, “the value of instantaneous spring constant ΔF/ΔD being stable” is simply expressed as “spring constant ΔF/ΔD”.

When measuring intraocular pressure with the measurement device, the following with FIGS. 11(a) to 11(c) explains the deriving of intraocular pressure after obtaining the spring constant ΔF/ΔD. Here, the intraocular pressure corresponds to the internal pressure of the measured object. In FIG. 11(a), the vertical axis is the intraocular pressure and the horizontal axis is the spring constant (ΔF/ΔD). In clinical research, the relationship between the intraocular pressure measured by a conventionally known device and the spring constant ΔF/ΔD measured by the measurement device of this embodiment is plotted, and an approximate expression representing the relationship between the two is determined by a statistical method. The mainstream of conventionally known intraocular pressure measurement devices is a type in which a probe is directly applied to the cornea.

The plots in FIG. 11 (a) are data from multiple human subjects. An approximate curve is obtained by a statistical method so that measurement results are improved for all subjects on average. The spring constant (ΔF/ΔD) is substituted into the function. In this approximate curve, the effect of rigidity that the eyeball-side layer of the eyelid and the surface of the eyeball have independently of intraocular pressure is improved on average for all subjects. The approximate curve may be linear instead of a curve.

FIG. 11(b) illustrates a creation of a calibration curve from data from one subject at various conditions of intraocular pressure. The values after applying the conversion illustrated in FIG. 11(a) are on the horizontal axis, and the values measured by a conventionally known intraocular pressure measurement device are on the vertical axis. In this way, individual variation in rigidity that the eyeball-side layer of the eyelid and the surface of the eyeball have independently of intraocular pressure is calibrated. Note that when plotting three or more points, the non-linearity is also calibrated.

FIG. 11(c) illustrates a direct calibration that does not perform the conversion in FIG. 11(a), where a calibration curve is created from the relationship between the stable value of the spring constant ΔF/ΔD and intraocular pressure measured by a conventionally known intraocular pressure measurement device. The rest are the same as FIG. 11(b).

For example, when the measurement device of this embodiment is owned by an individual and used for self-measuring intraocular pressure, since one's intraocular pressure changes even when the measured object is the same every time, the calibration described in FIG. 11(b) or 11(c) is effective.

Relative changes in intraocular pressure can be known by the spring constant (ΔF/ΔD) as the final output, even without performing the conversion in FIGS. 11(a) to 11(c).

By performing required information processing using these data, intraocular pressure can be measured by the measurement device.

That is, the measurement device can measure intraocular pressure by having a predetermined information processing means (for example, a microcontroller) process information that is given by the first sensing means and the second sensing means. The processing of information is described above using FIGS. 10 and 11. Algorithms, conversion formulas, and parameters required for the information processing described above using FIGS. 10 and 11 are stored in a storage unit (for example, a built-in non-volatile memory in the microcontroller) and referred to.

The above description is a case where the second sensing means senses the distance of movement D of the pressing portion in the direction of the measured object, when the contact surface is brought into contact with the surface of the measured object and the pressing portion is pressed toward the measured object.

In a case where the second sensing means senses the speed of movement of the pressing portion moving in the direction of the measured object, the distance of movement D can be calculated by integrating the sensed speed of movement, when the contact surface is brought into contact with the surface of the measured object and the pressing portion is pressed toward the measured object.

In a case where the second sensing means senses the acceleration of the pressing portion moving in the direction of the measured object, the distance of movement D can be calculated by double-integrating the sensed acceleration, when the contact surface is brought into contact with the surface of the measured object and the pressing portion is pressed toward the measured object.

In the measurement device of the embodiment described above, the first sensing means may be configured to sense the repulsive force continuously by bringing the pressing portion into contact with the first sensing means in the direction from the tip toward the rear end of the pressing portion facing the tip.

Various means can be adopted as the first sensing means if the first sensing means can continuously sense the repulsive force from the measured object by being in contact with the rear of the pressing portion, when the contact surface is brought into contact with the surface of the measured object and the pressing portion is pressed toward the measured object, For example, a force sensor, a touch-sensitive pressure sensor, a capacitance sensor, an electronic component available in the form of a load cell (strain gauge type, capacitive type, and the like), a strain gauge, or the like can be adopted as the first sensing means.

In the measurement device of the embodiment described above, the measurement portion can be a hollow structure that has an internal hollow portion inside. Examples of the hollow structure that has the internal hollow portion inside include a sealed structure, a hollow structure that has one or more holes for communicating between the internal hollow portion and the external space, and a hollow structure in which one or more breathable films are provided between the internal hollow portion and the external space, and the like.

As the hollow structure that has one or more holes for communicating between the internal hollow portion and the external space, a hollow structure that has one or more minute holes (through-holes) can be exemplified. An example of the hollow structure in which one or more breathable films are provided between the internal hollow portion and the external space is that a hollow structure that has one or more minute holes (through-holes) for communicating between the internal hollow portion and the external space where one or more breathable films (vent filters) for waterproof/dustproof performances are attached covering one or more minute holes (through-holes).

When the measurement portion is the hollow structure that has the internal hollow portion inside, as the structure that the pressing portion rear, which is the opposite side of the pressing portion from the tip, is supported by the measurement portion, a structure in which the pressing portion rear is supported on the outside of one wall face of the hollow structure that constitutes the measurement portion can be adopted. Specifically, structures such as (1) a structure in which the pressing portion is erected on the pressing portion rear on the outside of one wall face of the hollow structure, (2) a structure in which the pressing portion rear can be attached to/removed from the outside of one wall face of the hollow structure, and the like can be adopted.

When the measurement portion is the hollow structure that has the internal hollow portion inside, the first sensing means can be configured to sense the repulsive force continuously by sensing continuously the amount of bending of the one wall face toward the internal hollow portion by bringing the contact surface into contact with the surface of the measured object and pressing the pressing portion toward the measured object.

In addition, the first sensing means can be configured to sense the repulsive force continuously by sensing continuously the change in the internal pressure of the internal hollow portion generated by bending the one wall face toward the internal hollow portion by bringing the contact surface into contact with the surface of the measured object and pressing the pressing portion toward the measured object.

When the one wall face that constitutes the hollow structure and supports the pressing portion is a member that bends toward the internal hollow portion by the pressing (pressing operation), the first sensing means of the form described above can be adopted.

Further, when the measurement portion is the hollow structure that has the internal hollow portion inside, the first sensing means can be configured to sense the repulsive force continuously by sensing continuously the amount of movement of the one wall face toward the internal hollow portion by bringing the contact surface into contact with the surface of the measured object and pressing the pressing portion toward the measured object.

In addition, the first sensing means can be configured to measure the repulsive force continuously by measuring continuously the change in the internal pressure of the internal hollow portion generated by movement of the one wall face toward the internal hollow portion by bringing the contact surface into contact with the surface of the measured object and pressing the pressing portion toward the measured object.

When the one wall face that constitutes the hollow structure and supports the pressing portion is a member that has rigidity and does not bend toward the internal hollow portion by the pressing operation, the first sensing means of the form described above can be adopted.

For example, as the first sensing means, it is possible to continuously sense the amount of bending by adopting a variable resistor that is installed on the one wall face and the resistance of which changes according to the amount of bending of the one wall face. An example of a variable resistor is a strain gauge that allows for continuous measurement of the value of the resistance. As a position for installing the variable resistor, an inner surface of the one wall face facing the internal hollow portion can be considered. It is also possible to adopt a structure that the variable resistor is sandwiched between the pressing portion and the one wall face.

In addition, it is also possible to continuously sense the repulsive force by adopting a sensing means that continuously senses change in distance between the inner surface of the one wall face, which faces the internal hollow portion, and the other the inner surface, which is the opposite side of the internal hollow portion from the inner surface of the one wall face. The change in distance is caused by the one wall face bending toward the internal hollow portion. For example, an element whose electrical characteristics change according to the distance between one inner surface and the other inner surface can be adopted as the first sensing means. One example is a capacitance sensor configured by installing one electrode on the inner surface of the one wall face, which faces the internal hollow portion, and the other electrode on the other inner surface, which is the opposite side of the internal hollow portion from the inner surface of the one wall face. When the one wall face bends toward the internal hollow portion, the capacitance between the one electrode and the other electrode changes, which is continuously sensed to obtain the repulsive force.

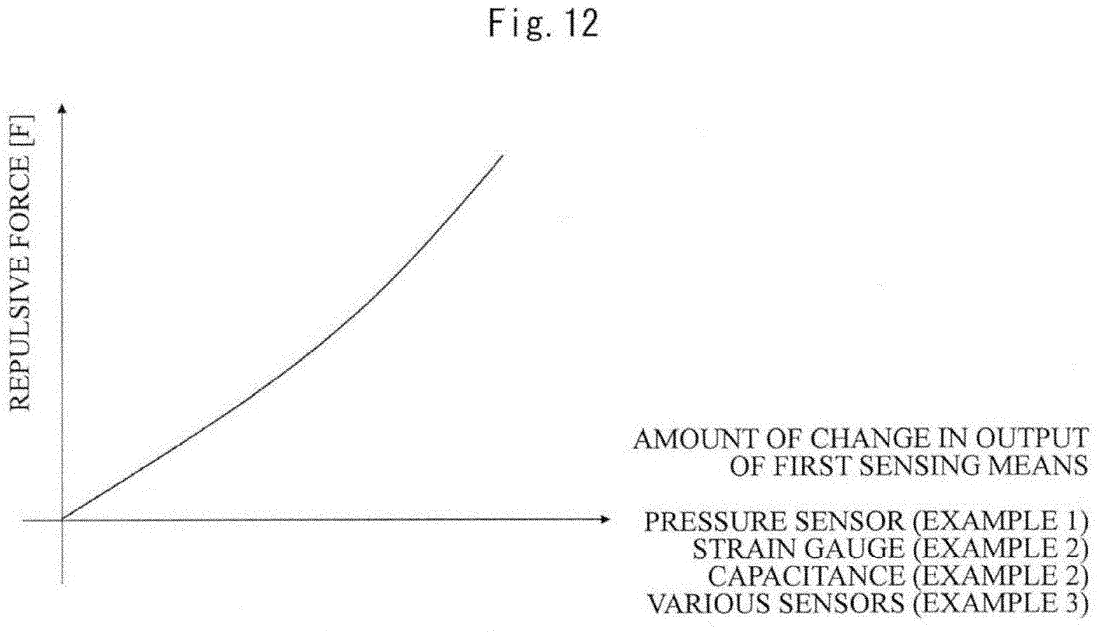

As illustrated in FIG. 12, the relationship between the output of the sensor used as the first sensing means and the repulsive force F is known in advance. The horizontal axis of FIG. 12 represents the amount of change in the output of the first sensing means, where the reference point is the output before the pressing operation. Then, based on the predetermined relationships and information known in advance, the information processing described above using FIGS. 10 and 11 is subsequently performed by a predetermined information processing means (for example, a microcontroller), and intraocular pressure can be calculated by the measurement device. Algorithms, conversion formulas, and parameters required for the information processing described above using FIGS. 10, 11, and 12 are stored in a storage unit (for example, a built-in non-volatile memory in the microcontroller) and referred to.

When a sealed structure or a hollow structure with one more minute holes and one or more vent filters are adopted as the hollow structure, the first sensing means can be configured to sense continuously the change in the internal pressure of the internal hollow portion generated by bending the one wall face toward the internal hollow portion by bringing the contact surface into contact with the surface of the measured object and pressing the pressing portion toward the measured object.

In this case, the physical quantity sensed by the first sensing means is the pressure P inside the sealed structure or the hollow structure provided with the minute hole and the vent filter described above. As illustrated in FIG. 12, the relationship between the output of the first sensing means (for example, a pressure sensor) and the repulsive force F is known in advance. This relationship differs depending on the shape and the member of the sealed structure or the like. And then, based on the relationships and information illustrated in FIG. 12, the information processing described using FIGS. 10 and 11 is subsequently performed by a predetermined information processing means (for example, a microcontroller), and intraocular pressure can be calculated by the measurement device. Algorithms, conversion formulas, and parameters required for the information processing described above using FIGS. 10, 11, and 12 are stored in a storage unit (for example, a built-in non-volatile memory in the microcontroller) and are referred to.

When continuously sensing the pressure P in the sealed structure or the internal hollow portion of the hollow structure provided with the minute hole and the vent filter described above, a structure in which the pressure sensor is placed inside the sealed structure or the like can be adopted as the first sensing means.

In FIG. 12, where the output of the first sensing means is converted into repulsive force F, a narrow section can be regarded as linear although a wide section appears non-linear. If the repulsive force can be measured with a very small amount of bending of the one wall face, the linear region of FIG. 12 can be used, which is desirable. Details will be later described in an example.

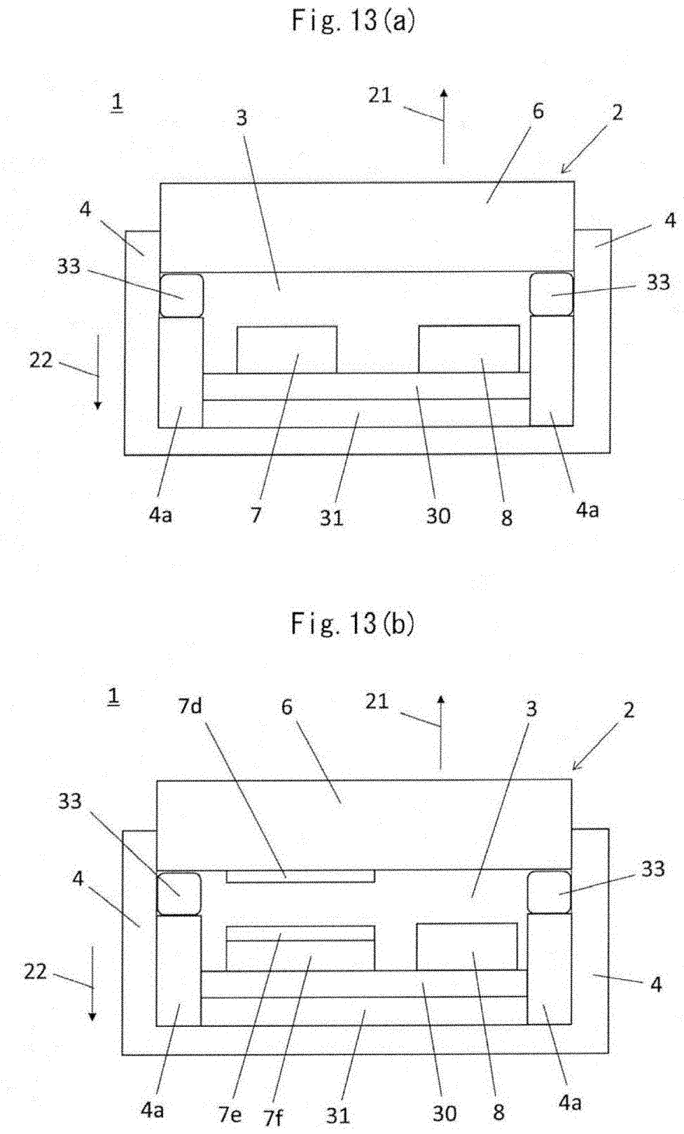

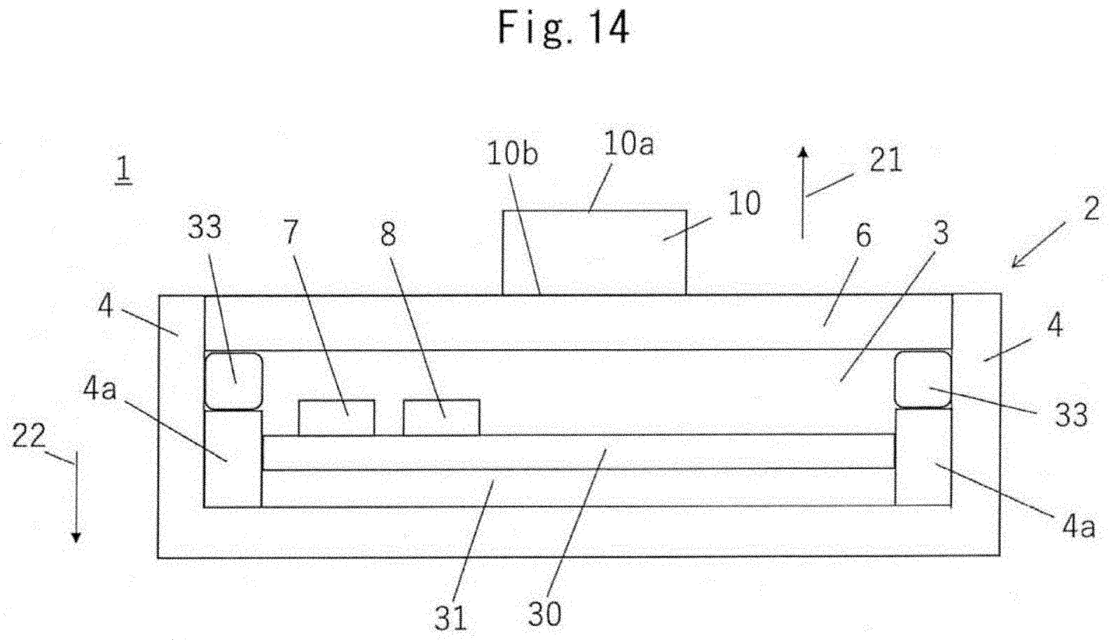

As described above, when the measurement portion has the internal hollow portion, the means of changing the pressure of the internal hollow portion is not limited to the bending of the one wall face. As in the embodiments explained later using FIGS. 13 and 14, the sealing part can be made elastic, in a way that the sealing part functions also as an elastic body. An O-ring, a gasket, a spring, a bellows structure, or the like are the examples of the elastic sealing means, where such elastic bodies are compressed and then change the pressure of the internal hollow portion.

When the one wall face that constitutes the hollow structure and supports the pressing portion is such a member that bends toward the internal hollow portion by the pressing operation, measurement range and accuracy can be optimized by adjusting the flexural rigidity and/or the size of the supporting area on the wall face of the measurement portion according to the approximate stiffness of the measured object such as food, parts of the body, or the like,

When the one wall face that constitutes the hollow structure and supports the pressing portion is such a member that has rigidity and does not bend toward the internal hollow portion due to the pressing operation, measurement range and accuracy can be optimized by adjusting the compressive stiffness of the elastic bodies that are compressed during the pressing operation such as an O-ring, a gasket, a spring, a bellows structure, or the like, according to approximate stiffness of the measured object such as food or parts of the body.

With the measurement device that has aforementioned configurations, the distance of movement of the pressing portion toward the measured object and the repulsive force from the measured object when the contact surface is brought into contact with the surface of the measured object and the pressing portion is pressed toward the measured object can be continuously sensed.

As a result, it becomes possible to obtain the repulsive force with respect to the distance of movement, and from this relationship, the internal pressure of the measured object, which corresponds to the rigidity of the measured object that is in contact with the contact surface of the pressing portion of the measurement device, is obtained.

That is, the internal pressure of the measured object, which is intended to be measured and is corresponding to the rigidity of the measured object, can therefore be measured by bringing the contact surface of the pressing portion of the measurement device into contact with the surface of the measured object and pressing the pressing portion toward the measured object.

In this way, if the measured object can be regarded as a spring, the measurement device can be applied not only to the measurement of the internal pressure but also to the measurement of the rigidity itself or the repulsive force itself per a certain unit of distance of movement. By adopting the spring constant (ΔF/ΔD) as the measurement result when it is stable during the continuous sensing, the influence of the stiffness and thickness of the outermost layer that covers the film on the surface of the contents from further outside (the outermost layer whose rigidity is negligible) can be reduced.

For example, it can be used to measure the ripeness of fruits and the like.

It is also conceivable to use the measurement device to measure the repulsive force of a board such as a ski or the like, of a racket, and of bed springs by using a large contact surface.

A tire pressure can also be measured easily.

Furthermore, it is possible to measure the internal pressure corresponding to the rigidity of a targeted measurement point of a human body. For example, the measurement device can be used for measuring intraocular pressure, or the stiffness of intraperitoneal organs, muscle, bone soft tissue, or the like with minimum invasiveness and an easy matter, which have been difficult to self-measure in the past.

When measuring intraocular pressure, it is measured as the internal pressure of the eyeball.

When the measurement device is used for intraocular pressure, the contact surface is brought into contact with the eyeball or the eyelid and the pressing portion is pressed toward the eyeball or eyelid. By doing so, the distance of movement of the pressing portion in the direction of the eyeball or eyelid and the repulsive force from the eyeball, when the pressing portion is pressed, are continuously sensed, and intraocular pressure as the internal pressure of the eyeball can be measured.

The measurement device finds the stable value of instantaneous ΔF/ΔD during the continuous sensing as described above, which is referred to when internal pressure of the measured object is calculated.

Hence, although the skin-side layer of the eyelid (the outermost layer whose rigidity is negligible) affects the instantaneous spring constant at the start of pressing, adopting the stable instantaneous spring constant during the press for the calculation of intraocular pressure, the influence of the layer on the measurement is reduced.

Since intraocular pressure can be measured through the eyelid as described above, it is possible to provide a non-invasive intraocular pressure measurement device without drug administration or the like.

In addition, by using components made into integrated circuit (IC) packages as the first sensing means and the second sensing means, it is possible to provide a very small measurement device that can be easily used by anyone and has excellent portability. Therefore, it becomes possible for the subjects to self-measure their intraocular pressure.

Since the measurement device is small, it can be fixed to one or more fingers with a belt/adhesive tape or can be combined with a holding means that has a structure such as a finger cot. It is possible to provide an intraocular pressure measurement device that is operated similarly to palpation by a person who measures intraocular pressure, by inserting a finger into a finger cot-shaped holding means to hold the measurement device.

### Example 1

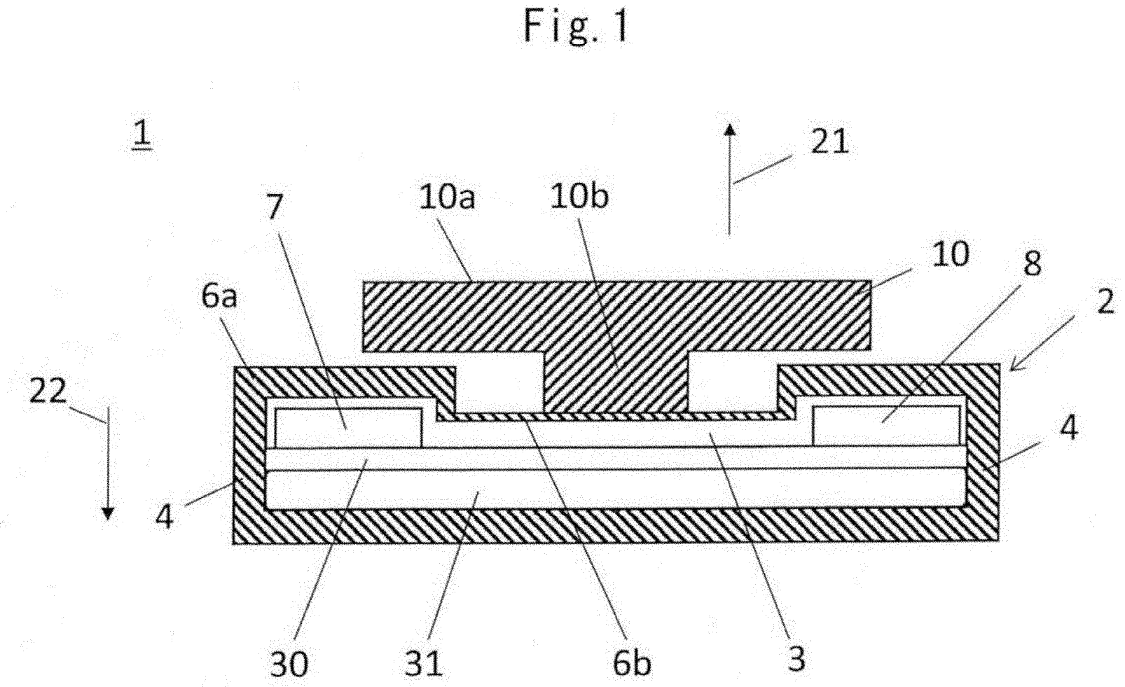

FIG. 1 illustrates an embodiment of the measurement device of the present invention that is used as an intraocular pressure measurement device.

The measurement device 1 illustrated in FIG. 1 includes a pressing portion 10 and a measurement portion 2. In the illustrated example, the measurement portion 2 includes a first sensing means 7 and a second sensing means 8.

The pressing portion 10 is provided at the tip with a contact surface 10a (upper end in FIG. 1) that comes into contact with the eyelid, which is the measured object.

In the illustrated embodiment, the measurement portion 2 is a hollow structure that has an internal hollow portion 3 inside. The hollow structure is a sealed structure.

It is also possible to configure a hollow structure that has one or more holes, for example, one or more minute holes (through-holes) for communicating between the internal hollow portion 3 and the external space of the measurement portion 2. The hollow structure can have one or more breathable film between the internal hollow portion 3 and the external space of the measurement portion 2, which will be described later.

In the embodiment illustrated in FIG. 1, a pressing portion rear 10b, which is the opposite side of the pressing portion 10 from the tip that has the contact surface 10a, is erected on the outside of one wall face of the measurement portion 2 that has a sealed structure.

Besides the structure of the pressing portion 10 and the measurement portion 2, various structures can also be adopted. For example, it is possible to adopt a structure in which the pressing portion 10 is movably supported by the measurement portion 2 so that the pressing portion 10 can move from the tip that is provided with the contact surface 10a toward the pressing portion rear 10b, or as one example thereof, a structure in which the pressing portion rear 10b is slidably supported by the measurement portion 2 so that the pressing portion 10 can move in the direction from the tip of the pressing portion 10 toward the rear of the pressing portion 10. In addition, it is also possible to adopt a structure in which the pressing portion 10 and the measurement portion 2 are separate and the pressing portion rear 10b of the pressing portion 10 is attachable to/removable from the measurement portion 2. These will be described later.

In the embodiment illustrated in FIG. 1, one wall face of the measurement portion 2 with the pressing portion rear 10b erected on the outside has a stepped structure, where the central portion 6b in the radial direction is recessed more than the outer peripheral portion 6a in the radial direction. Hereinafter, the outer peripheral portion 6a in the radial direction and the central portion 6b in the radial direction may be represented as “wall face 6”, meaning the one entire wall face of the measurement portion 2

The first sensing means 7 and the second sensing means 8 are mounted on a substrate 30 that is on a battery 31 and are placed in the internal hollow portion 3.

The first sensing means 7 is a sensing means that continuously senses the repulsive force F from the eyeball when the contact surface 10a is brought into contact with the eyeball or eyelid and the pressing portion 10 is pressed toward the eyeball or eyelid.

Note that, when the contact surface 10a is brought into contact with the eyelid and the pressing portion 10 is pressed, the eyelid may be closed.

In the illustrated example, the contact surface 10a is brought into contact with the eyeball or eyelid, and the pressing portion 10 is pressed toward the eyeball or eyelid in the direction indicated by arrow 21. As a result, the wall face 6 bends toward the internal hollow portion 3 in the direction indicated by arrow 22, thereby changing the internal pressure of the internal hollow portion 3. A sensing means that continuously senses change in internal pressure can be adopted as the first sensing means 7. For example, a pressure sensor can be adopted as the first sensing means 7.

The second sensing means 8 is a sensing means that continuously senses distance of movement D of the pressing portion 10 toward the eyelid when the contact surface 10a is brought into contact with the eyeball or eyelid and the pressing portion 10 is pressed toward the eyeball or eyelid.

For example, an accelerometer can be adopted as the second sensing means 8.

As the first sensing means 7 composed of the pressure sensor and the second sensing means 8 composed of the accelerometer, components integrated into one or more IC-packages can be used and placed on the substrate 30.

The intraocular pressure measurement using the measurement device 1 is performed as follows.

The contact surface 10a of the pressing portion 10 is brought into contact with the eyeball or eyelid, and the measurement device 1 is pressed toward the eyeball or eyelid.

For example, a holding portion that a human finger can be inserted to/removed from is attached to the bottom face of the measurement portion 2 that has a sealed structure in FIG. 1, while holding the measurement device 1 by inserting the finger therein, the measurement device 1 is pressed toward the eyelid as indicated by arrow 21.

Alternatively, a pressing device is separately prepared, and while holding the measurement device 1 by the pressing device, the measurement device 1 is pressed toward the eyelid as indicated by arrow 21. For example, a pressing device (not illustrated in the drawings) in which the measurement device 1 is slidably supported is fixed above/below/around the eyes of the subject by using a human's left hand. Then, the pressing portion 10 in which the contact surface 10a is in contact with the eyeball or eyelid of the subject is pressed in the direction of the eyeball or eyelid by the pressing operation by the pressing device.

The pressing operation described above may be performed by a right hand of a measurer who has the pressing device fixed around the eyes of the subject. In addition, it is possible for a subject to fix the pressing device around his/her eyes with his/her own left hand and perform the pressing operation with his/her right hand.

The location where the contact surface 10a is brought into contact may be any location as long as the contact surface 10a is perpendicular to the normal direction of the eyeball.

When the pressing portion 10 is pressed toward the eyeball or eyelid, the central portion 6b of the wall face 6 in the radial direction bends toward the internal hollow portion 3. The first sensing means 7 continuously senses the change in the internal pressure of the internal hollow portion 3 caused by the bending.

At the same time, the distance of movement D of the pressing portion 10 in the direction of the eyelid when the pressing portion 10 is pressed toward the eyeball or eyelid is continuously sensed by the second sensing means 8.

The data sensed by the first sensing means 7 and the second sensing means 8 is sent to an information processing means (for example, a microcontroller) (not illustrated in the drawings), in which predetermined information processing is performed.

From the change in the internal pressure P of the internal hollow portion 3 sensed by the first sensing means 7, the repulsive force F from the eyeball to which the contact surface 10a of the pressing portion 10 is in contact is continuously calculated. An information processing means (for example, a microcontroller) (not illustrated in the drawings) performs the calculation referring to predetermined conversion formulas and parameters illustrated in FIG. 12. The conversion formulas and parameters are stored in a non-volatile memory (not illustrated in the drawings).

The distance of movement D of the pressing portion 10 toward the eyelid is continuously calculated from acceleration sensed by the second sensing means 8 constituted by the accelerometer.

Placing the measurement device 1 in a stationary position at the start of pressing, the acceleration of gravity applied to each axis of X, Y, and Z of the accelerometer is set as a reference value, and the differences from the acceleration reference value are continuously recorded for each axis after the start of pressing. As a result, the distance of movement D of the pressing portion toward the measured object can be calculated by excluding the gravity component and considering only the acceleration generated by the pressing operation.

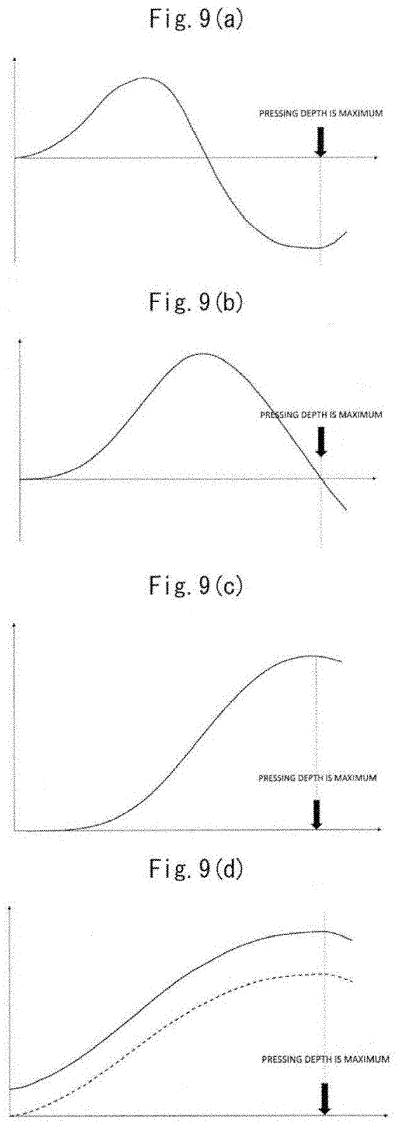

In FIG. 9(a), X-axis is the time, and Y-axis is the accelerometer output: a. The accelerometer constitutes the second sensing means 8 that is placed in or on the measurement device 1. The time point when the output of the pressure sensor changes by a certain value or more is set as t=0.

It is preferred for the contact surface 10a to be pressed in a state where the contact surface 10a is in contact with the eyelid or the eyeball with a strength that does not deform the eyeball at the start of pressing in order to model the rigidity as a linear spring.

By integrating the relationship illustrated in FIG. 9(a), the relationship between time on X-axis and speed of movement: v of the measurement device 1 on Y-axis is obtained as illustrated in FIG. 9(b).

Further, by integrating the relationship illustrated in FIG. 9(b), that is, by double integrating the relationship illustrated in FIG. 9(a), the relationship between time on X-axis and distance of movement: D of the measurement device 1 on Y-axis is obtained as described in FIG. 9(c).

The calculation is performed also by the information processing means (for example, a microcontroller) (not illustrated in the drawings).

Subsequently, the information processing means (for example, a microcontroller) (not illustrated in the drawings) calculates the stable value of instantaneous ΔF/ΔD during the continuous sensing, based on the repulsive force F from the eyeball and the distance of movement D of the measurement device 1 toward the eyelid that are continuously sensed. The stable value of instantaneous ΔF/ΔD during the continuous sensing is in other words, the value of ΔF/ΔD that represents the internal pressure of the measured object with reduced influence of the skin-side layer of the eyelid (the outermost layer whose rigidity is negligible). This concept is illustrated in FIGS. 10(a) and 10(b).

Then, as explained referring to FIGS. 11(a)-11(c) in above-described embodiment, the intraocular pressure is derived from the relationship between the intraocular pressure measured by the conventionally known intraocular pressure measurement device in the clinical study and the stable value of instantaneous ΔF/ΔD during the continuous sensing.

This information processing is also performed by an information processing means (for example, a microcontroller) (not illustrated in the drawings) by referring to predetermined conversion formulas and parameters that are stored in a non-volatile memory (not illustrated in the drawings) and are illustrated in FIGS. 11(a)-11(c).

The intraocular pressure that is thereby derived by information processing by the information processing means (for example, a microcomputer) (not illustrated in the drawings) can be displayed by, for example, a display means placed in or on the measurement device 1 (not illustrated in the drawings).

Alternatively, a wireless communication means may be placed in or on the measurement device 1, and the intraocular pressure derived by information processing by the information processing means may be displayed on a display terminal possessed by a person who measures the intraocular pressure using the measurement device 1.

The result may be output by an acoustic component such as a speaker or a buzzer placed in or on the measurement device 1 (not illustrated in the drawings). Voice feedback enables measurers to know the result of success/failure of the pressing operation, the cause of the pressing operation failure, and/or the measurement result. Voice feedback may be performed from the display terminal.

It is also possible to extend sealed wires (not illustrated in the drawings) from the measurement portion 2 while sealing the measurement portion 2 by a sealed structure, and display and output the intraocular pressure by an external device of the measurement portion 2.

Power for driving the first sensing means 7, the second sensing means 8, the information processing means (for example, a microcontroller) (not illustrated in the drawings), the wireless communication means (not illustrated in the drawings), and the like described above is supplied from the battery 31. Alternatively, those components may be driven by power wirelessly supplied from the outside without using a battery.

The information processing means described above (for example, a microcontroller), wireless communication means, battery, and the like can also be placed outside the measurement portion 2. It is also possible to make the pressing portion 10 in a hollow structure and place the battery inside the pressing portion 10. The hollow portion inside the pressing portion 10 may be connected to the internal hollow portion 3, and the battery may be placed in the connecting portion. By thereby placing the battery in a place that makes the measurement device 1 thinner, operability of the pressing operation improves, and the accuracy for obtaining the distance of movement D of the pressing portion 10 toward the measured object by using the accelerometer improves. A battery may be placed in the internal hollow portion 3 of the measurement portion 2.

A pressure sensor made by attaching a tubular body, a cylinder body, a cylindrical body, or the like to an IC package may be used. A pressure sensor may be placed outside the internal hollow portion 3 if a tubular path for transmitting the pressure of the internal hollow portion 3 to the pressure sensor can be formed.

Even when the components are placed in the internal hollow portion 3 of the measurement portion 2, the measurement device 1 can be extremely small by using the components integrated into one or more IC packages.

When calculating the distance by double integration of acceleration, the following situations are preferred.

(1) The S/N ratio of acceleration is high.

(2) The DC (offset) component of acceleration is small.

(3) The integration time is short.

When the measurement device 1 is made smaller, the operability improves, and it becomes possible to perform the pressing operation even by one or more fingers of the measurer. When the measurement device 1 is small, the measurer can freely perform the pressing operation, so that the preferred conditions described above can be satisfied.

By limiting the time of movement of the pressing portion from the start of pressing to the deepest point in the direction of arrow 21 to a short time within 300 milliseconds, preferably within 200 milliseconds, when measuring the distance by double-integration of the acceleration, an error in distance measurement due to the integration of acceleration noise (mainly DC component) is reduced.

Because the measurement device 1 is small, the measurement device 1 can be combined with a structure like a finger cot, thereby enabling the subject to use the measurement device 1 as if palpation is performed by himself/herself, and therefore the operability improves. This allows for the pressing operation in a short time within 300 milliseconds, preferably within about 200 milliseconds as described above, which is advantageous for the distance measurement by double-integration of acceleration.

Further, by setting the timing of starting the integration to the time when a certain value of the change is seen in the output of the first sensing means 7, the integration time can be minimized and the error in distance measurement can be reduced.

When measuring intraocular pressure through the eyelid, the distance of movement D is continuously sensed from a state where the contact surface 10a is in contact with the eyelid as a pressing start position of the pressing portion 10 until the distance of movement D (pressing depth) of the pressing portion 10 is maximized. Intraocular pressure can preferably be measured with a short distance of movement so as not to damage the eyeball.

When the present invention is implemented as an intraocular pressure measurement device, movement about 1 mm can be measured by double-integration of the accelerometer output. Even if there is an error in the absolute value of the measured distance of movement, the problem is alleviated for the following reasons.

(1) Even if the distance of movement D of the pressing portion 10 toward the measured object is larger or smaller than the actual distance for all measurers, such a tendency is calibrated by the conversion illustrated in FIG. 11(a). It is also valuable to know the daily relative changes in intraocular pressure as the results of the measurement device 1, which does not require absolute accuracy.

(2) If there is a tendency in the error of the distance of movement D of the pressing portion 10 toward the measured object due to an operation habit of a certain measurer, it is calibrated by the conversion in FIG. 11(b) or FIG. 11(c).

It is preferred that the accelerometer adopted as the second sensing means 8 should output three-dimensional acceleration for the following reasons.

(1) Even if the X-Y-Z axes of the accelerometer and the X-Y-Z axes of the measurement device 1 deviate from each other, the direction perpendicular to the contact surface 10a, which is the direction of the press, can be expressed by a composite vector consisting of multiple axes of the accelerometer output.

(2) As will be described later, even if the pressing direction shifts during the press or the measurement device 1 rotates, it is possible to perform compensation or determine such pressings as pressing operation failure.

(3) The pressing device described above makes a pressing direction adjustment easy as descried below.

The only acceleration that is output by the accelerometer when stationary is the acceleration of gravity. When the three-dimensional acceleration is sensed by the accelerometer used in the second sensing means 8, tilt angle in the three-dimensional space of the measurement device 1 before a pressing operation can be known. If the posture of the subject is known in advance, the angle of the measurement device 1 can be oriented to correspond to the subject's posture, so that the pressing operation in the appropriate direction becomes easy.

When the accelerometer is mounted in parallel to the substrate 30 as illustrated in FIG. 1 and all the axes have no offset, the Z axis of the accelerometer generally corresponds to the direction perpendicular to the contact surface 10a. Hereinafter, description will assume that the direction perpendicular to the contact surface 10a is the Z axis of the accelerometer output.

It is desirable to bring the contact surface 10a into contact with the appropriate location at the appropriate angle and press the measurement device in the appropriate direction. If the location and the angle are not ideal, the contact surface 10a moves translationally in a diagonal direction, and a non-negligible output appears on the X and Y axes of acceleration. There are two types of situations as follows. Here, rotation component is ignored. Rotation compensation will be described later.

(1) A case where the contact angle of the contact surface 10a is deviated but the pressing direction is the normal direction (toward the center) of the eyeball.

(2) A case where the contact angle of the contact surface 10a is appropriate but the pressing direction deviates from the normal direction of the eyeball.

The component of the repulsive force vector perpendicular to the contact surface 10a, which bends the wall face 6, is the repulsive force sensed by the first sensing means 7 constituted by the pressure sensor. If the direction perpendicular to the contact surface 10a and the Z-axis direction of the accelerometer are the same, in both cases (1) and (2), similar to the case when there is no deviation in the pressing direction, it is possible to determine the spring constant ΔF/ΔD from the relationship between the distance of movement D of the pressing portion 10 toward the measured object that is calculated by the output of only Z-axis of the accelerometer, and the repulsive force F, which is sensed by the first sensing means 7 constituted by the pressure sensor. In case of (2), the repulsive force from the normal direction of the eyeball is all applied in the direction perpendicular to the contact surface 10a, so the same calculation as in case (1) can be applied. In both cases (1) and (2), large deviation angles may be regarded as pressing operation failure. In order to know that such a pressing operation has been performed, for example, whether the distance of movement of the accelerometer in X-axis or Y-axis direction is larger than a certain threshold may be used as a determination criterion.

A pressing operation that starts from a state where the contact surface 10a is not in contact with anything, thereby being hit on the measured object rather than pressed may be determined as a pressing failure. The determination may be made by focusing on the high-frequency component of the acceleration. In such a case, a high-frequency acceleration, which expresses an impact, is observed at the moment when the contact surface 10a hits the eyelid. If the pressing portion 10 is pressed in the direction of the eyelid while the contact surface 10a is in contact with the eyelid, there is a limit on the high-frequency component of the acceleration output because the eyelid and the eyeball function as a cushion (mechanical low-pass filter) and because there is a limit on the maximum value of the acceleration output that normal pressing operations can generate. To determine an unfavorable pressing operation as a pressing failure, the high-frequency component needs to be measured as well, so accelerometers with high data rate and wide frequency bandwidth are preferred. Other unfavorable pressing operations and movements of the pressing portion 10 can also be discerned, based on the sensed acceleration waveform, pressure waveform, or rotational movement during the press sensed by a third sensing means that is described later. Such a pressing operation may be regarded as a failure and measurement may be performed again.

Although not illustrated in the drawings, it is possible to configure the measurement portion 2 further including a third sensing means constituted by a gyroscope or the like that senses a rotational movement that occurs when the pressing portion 10 is pressed in the direction of the eyelid while the contact surface 10a is in contact with the eyelid.

When the contact surface 10a of the pressing portion 10 is brought into contact with the eyelid and pressed in the direction of arrow 21, if rotation occurs immediately before or during the press, the direction of gravity on the second sensing means 8 constituted by the accelerometer changes, which may cause an error in distance measurement. By processing the output from the third sensing means constituted by a gyroscope or the like by the information processing means (for example, a microcontroller) (not illustrated in the drawings), such rotation can be compensated even if it occurs.

To compensate the rotation, it is preferred that the second sensing means 8 is an accelerometer with three-dimensional output and also the third sensing means is a gyroscope with three-dimensional output, because the rotation in any direction in a three-dimensional space can be compensated. To form three dimensions, one sensor that has three axes may be adopted, or a plurality of sensors may form three axes. In the following, a method of compensating an error in distance measurement due to a change in the direction of gravity applied to the measurement device 1 due to rotation will be exemplified, assuming a X-Y-Z 3-axes accelerometer and gyroscope is used.

The movement of the pressing portion 10 during the press is decomposed into translational movement and rotational movement. First, in a state where the measurement device 1 is in stationary immediately before the start of pressing (time to), the tilt angle of the measurement device 1 in the three-dimensional space is determined by the accelerometer. Let the acceleration output of gravity then be gt₀. In stationary, the accelerometer outputs only the acceleration of gravity, hence the three-dimensional angle of the measurement device 1 can be measured from the accelerometer output.

The output of each axis of X, Y, and Z of the gyroscope that is adopted as the third sensing means is continuously recorded during the press. Next, the axis and angle of rotation of the measurement device 1 in the three-dimensional space in Δt, from t₀ to t₁ where to is the time at the start of pressing, is calculated only from the gyroscope output. From the recorded gyroscope output and the angle of the measurement device 1 at the time to known in advance, the angle of the measurement device 1 at the time t₁ can be calculated. The calculation is repeated based on the updated angle. The calculation continues and the calculation results are recorded at least until time (tₙ) when the spring constants ΔF/ΔD stabilize. If the angle at each interval of time is calculated only from the gyroscope output, the amount of the accelerometer output contributed by gravity on each axis at each time interval can be calculated. Subtracting the amount from the acceleration output of each axis during the press, the acceleration of each axis excluding the contribution by rotation and gravity, which is the acceleration that contributes to the distance of movement D of the pressing portion 10 toward the measured object, is obtained. Here, the idea that the rotation of a rigid body in the three-dimensional space in a very short interval of time can be regarded as a rotation about one axis of an X-Y-Z composite vector is based on Euler's rotation theorem. If the rotation angle is large, the pressing operation may be regarded as a pressing operation failure and the pressing may be performed again.

Note that, at present, a X-Y-Z 3-axis accelerometer and a X-Y-Z 3-axis gyroscope in one IC package is commercially available. Such an IC package can also be adopted as the second sensing means 8 and the third sensing means. Then, the measurement device 1 can be formed in a small size.

A user interface for operating the measurement device 1 can be configured by using any one or a plurality of the first sensing means 7, the second sensing means 8, and the third sensing means. For example, alternative functions to general input methods such as push buttons capacitance sensors can be installed, such as turning on the power when the measurement device 1 is double-tapped (using accelerometer), performing wireless communication with a display terminal when the measurement device 1 is rotated by 45 degrees about the Z axis and rotated backward and repeating it twice (using gyroscope), and displaying measurement data history when the pressing portion 10 is pressed three times with shorter intervals than intraocular pressure measurements (using pressure sensor).

The contact surface 10a that comes into contact with the eyelid can have a concave shape that fits the eyelid's convex shape. When measuring internal pressure of the measured object by the measurement device 1, the contact surface 10a desirably has a shape that best fits the shape of the measured object.

Atop view of the measurement portion 2 and the pressing portion 10 in the embodiment of FIG. 1 are both circular shapes, and the overall shape of the measurement device 1 is cylindrical. The measurement portion 2 can be 10 mm to 20 mm in outer diameter and 2 mm to 10 mm in height.

The thickness of the pressing portion 10 and the height (vertical size) of the pressing portion rear 10b are preferably set such that when the contact surface 10a is brought into contact with the eyelid and the measurement device 1 is pressed in the direction of arrow 21, the outer peripheral portion 6a in the radial direction does not touch the periphery of the eyelid.

In the embodiment illustrated in FIG. 1, the central portion 6b in the radial direction is recessed more than the outer peripheral portion 6a in the radial direction (stepped structure). The pressing portion rear 10b is erected on the outside of the central portion 6b, which reduces the height and thickness in vertical direction of the structure of the measurement device.

The top view shape of the measurement portion 2 and the pressing portion 10 in the embodiment of FIG. 1 do not have to be circular.

The entire measurement portion 2 can be made of, for example, plastic. As described above, the central portion 6b where the pressing portion 10 is erected has a structure such that when the pressing portion 10 is pressed in the direction of arrow 21 in a state where the contact surface 10a is in contact with the eyelid, the central portion 6b bends toward the internal hollow portion 3 in the direction of arrow 22 according to the repulsive force from the eyeball.

Therefore, it is desirable that the central portion 6b of the wall face 6 has a thin plate shape with a small thickness so that the central portion 6b can bend.

To make the wall face 6 flexible, the area of the pressing portion rear 10b may be made smaller than the area of the contact surface 10a so that the area where the pressing portion 10 is in contact with the wall face 6 is reduced as illustrated in FIGS. 1, 3, and 4.

In the embodiment illustrated in FIG. 1, the wall thickness of the central portion 6b is made smaller than the wall thickness of the outer peripheral portion 6a.

The entire wall face 6 can be as thick as the central portion 6b.

However, if the bending of the wall face 6 toward the internal hollow portion 3 in the direction of arrow 22 is large, the calculation of the distance of movement D of the pressing portion 10 toward the eyelid sensed by the second sensing means 8 is affected.

The difference between the distance of movement of the measurement portion 2 toward the measured object sensed by the second sensing means 8 and the bending distance of the wall face 6 toward the internal hollow portion 3 is the distance of movement of the pressing portion 10 toward the measured object, when pressed.

By adopting the first sensing means 7 and the second sensing means 8 that are made into an IC-package, the internal hollow portion 3 can be made extremely small, and even if the amount of bending of the wall face 6 is very small, it is possible to generate a change in the internal pressure of the internal hollow portion 3 required to sense a change in the repulsive force F.

Further, since the amount of bending of the wall face 6 can be very small, the second sensing means 8 can be placed on the substrate 30 instead of on the pressing portion 10.

Since the change in the repulsive force F can be sensed even if the amount of bending of the wall face 6 is very small, a deep press is not required to improve the accuracy of the repulsive force F. This contributes to non-invasiveness when the measurement device 1 is used as an intraocular pressure measurement device.

There is another advantage in the small amount of bending of the wall face 6. In FIG. 12, the relationship is linear near the origin, but the deeper the press, the more non-linear. The factors contributing to non-linearity are considered to be the characteristics of the member of the wall face 6 and the force from the increased pressure in the internal hollow portion 3 pushing the wall face 6 back during the pressing operation, the both of which can be handled by the conversion illustrated in FIG. 12. However, in this example, the internal hollow portion 3 is small, and thus even if the amount of bending of the wall face 6 is very small, a change in the internal pressure of the internal hollow portion 3 that is large enough to be sensed by the first sensing means 7 is generated. If the amount of bending is very small, the influence of the characteristics of the member of the wall face 6, which is one of the causes of the non-linearity in FIG. 12, can be reduced. To suppress the influence of the force from the increased pressure of the internal hollow portion 3 pushing the wall face 6 back during the pressing operation, and to use the linear region near the origin in FIG. 12, it is preferred that the repulsive force can be sensed with as small internal pressure change as possible. Therefore, the first sensing means 7 desirably has low noise. If the linear region near the origin in FIG. 12 can be used in the period from the start of pressing to the end of pressing where the pressing depth is maximum, the conversion using FIG. 12 becomes simple. The flex of the wall face 6 can be selected considering the material or thickness of the wall face 6.

As in this example, when the first sensing means 7 for continuously sensing the repulsive force from the eyeball according to the movement of the pressing portion 10 in the direction of arrow 21 is constituted by the pressure sensor placed in or on the internal hollow portion 3, it is desirable that the structural parts other than the wall face 6 (bottom face and cylindrical wall face 4) are rigid to sense accurately and reliably the pressure change by the first sensing means 7 constituted by the pressure sensor. For example, the bottom face and the cylindrical wall face 4 can be made thicker than the outer peripheral portion 6a.

Adopting the rigid structure described above is advantageous because the conversions in FIGS. 11 and 12 are simplified.

In addition, in a case where a holding means such as a finger cot is attached under the measurement device 1 in FIG. 1 and the subject inserts his/her finger into the finger cot-shaped holding means, and presses the measurement device 1 toward the eyeball or eyelid by himself/herself, it is also advantageous to adopt such a rigid structure.

Here, force is exchanged between the finger and the contact surface of the finger cot-shaped holding means, and between the wall face 6 and the eyeball or eyelid through the pressing portion 10. Then, if such a rigid structure is adopted, the exchange of force between the finger and the contact surface of the finger cot-shaped holding means can be completely ignored, and only the amount of bending of the wall face 6 among multiple wall faces that constitute the sealed structure, can be regarded to contribute to the pressure change in the internal hollow portion 3.

The wall face 6 can be made of, for example, plastic.

Note that the flexural rigidity of the wall face 6 is affected by the temperature and the pressure difference between the internal hollow portion 3 and the outside.

To improve the accuracy when calculating the repulsive force F by an information processing means (for example, a microcontroller) (not illustrated in the drawings) from the sensed data by the first sensing means 7 constituted by the pressure sensor, the output of a temperature sensor (not illustrated in the drawings) and/or a pressure sensor for the external pressure (not illustrated in the drawings) can be saved in a memory before measuring intraocular pressure and referred to during the information processing so that the temperature and/or the pressure difference between the internal hollow portion 3 and the outside can be taken into account for compensation.

When the flexural rigidity of the wall face 6 has a temperature dependence, performing a temperature compensation is preferred. Instead of measuring the surface temperature of the wall face 6, the temperature sensor for compensation (not illustrated in the drawings) may be mounted on the substrate 30, or an output of a built-in temperature sensor in the first sensing means 7 or the second sensing means 8 (when there is a built-in temperature sensor) may be read out and used for the compensation.

Another conceivable factor that affects the flexural rigidity of the wall face 6 is a pressure difference between the internal hollow portion 3 and the outside that is existing before the press. Such a situation is when the internal hollow portion 3 is sealed, and the altitudes of manufacture and use are significantly different or an ambient pressure changes greatly due to weather, or the like. A sensor that outputs a differential pressure from the ambient pressure (gauge pressure sensor) may be used as the first sensing means 7 for compensation by always knowing the differential pressure between the internal hollow portion 3 and the outside. When adopting a gauge pressure sensor, sensing area of the gauge pressure sensor is required to be exposed to both the internal hollow portion and the outside but another absolute pressure sensor is not required to measure the ambient pressure outside.

In addition, for a situation where the pressure difference between the internal hollow portion 3 and the outside during a measurement is large, a hole and a plug may be provided in the measurement portion 2 in order to make the pressure difference negligible.

The plug may be made of a rubber material so that the measurement portion 2 can be sealed when the plug is attached to the measurement portion 2. By removing the plug and attaching it again before the measurement, the influence of the pressure difference between the internal hollow portion 3 and the ambient pressure on the flexural rigidity of the wall face 6 can be reduced to a negligible level in the calculation of the repulsive force.

By providing a ventilation through a minute hole (through-hole) in the measurement portion 2 that is sealed, the pressure of internal hollow portion 3 and the outside are equalized before using the measurement device 1. Thus, the influence on the flexural rigidity of the wall face 6 caused by the pressure difference between the internal hollow portion 3 and the ambient pressure before the pressing operation can be ignored.

A film (vent filter) may be attached covering the through-hole to provide waterproof/dustproof performances. Then, the measurement device 1 can be waterproof/dustproof, removing the necessity of consideration of the influence on the flexural rigidity of the wall face 6 caused by the pressure difference between the internal hollow portion 3 and the ambient pressure that exists before the pressing operation.

In either case where only the through-hole is used or where the vent filter is further added, the measurement portion 2 does not have a completely sealed structure and has a ventilation. This ventilation configures a mechanical high-pass filter on a rising pressure waveform illustrated in FIG. 9(d), and it becomes the dotted line waveform. By shortening the period from the start of pressing to the end (depth is maximum) of pressing, the internal pressure changes before the gas in the internal hollow portion leaks, therefore the effect of the ventilation on the continuous sensing of repulsive force is reduced. Even if there is an error in the conversion illustrated in FIG. 12 due to the ventilation, the error has a tendency, so the error is calibrated by the conversion in FIGS. 11(a)-11(c).

When the measurement portion 2 that has the sealed structure is made to have the ventilation, a pressure sensor that outputs an absolute pressure may be adopted as the first sensing means 7 and may be placed inside the internal hollow portion 3. A sensor that outputs differential pressure based on the vacuum (0 hPa) is defined as the absolute pressure sensor. If the IC-packaged microelectromechanical systems (MEMS) are used for the absolute pressure sensor as the first sensing means 7, for the accelerometer as the first sensing means 8, and for the gyroscope as the third sensing means, the measurement device 1 can be configured to be compact at a low cost.

The first, second, and third sensing means may each be constituted by multiple sensors and the outputs of which may be averaged to reduce noise.

When providing the ventilation to the measurement portion 2 that has the sealed structure, the ventilation rate may be selected by the size of the through-hole or by the type of the vent filter.

All parts of the sealed structure except the ventilation may be sealed by means of adhesive, welding, O-ring, gasket, or the like.

When the accelerometer and the absolute pressure sensor are adopted as in this example, an activity monitor for movement also in vertical direction can be added on the intraocular pressure measurement device as an add-on. When the measurement device 1 is used to know the daily fluctuation of intraocular pressure, the user of the measurement device 1 carries the measurement device 1 when going out. Thus, the intraocular pressure measurement device in a very small size with excellent portability and the activity monitor are a good match.

When the measurement device 1 is carried directly being put in a user's pocket without a case, the pressure sensor output fluctuates unintentionally due to the deformation of the internal hollow portion 3, which is an error factor for the purpose of measuring the movement in vertical direction as the amount of activity. Hence, the rate of ventilation in the hole and the vent filter installed in the measurement portion 2 is preferably high. When the rate of ventilation is high, the time until the internal and the external pressure equalize is short.

However, the rate of ventilation is preferably low to sense the repulsive force from the measured object. When the rate of ventilation is small, the S/N ratio of the pressure waveform illustrated in 9(d) is improved, the bandwidth of low-frequency portion of the pressure waveform is expanded, and the sensing of the repulsive force becomes accurate. An appropriate rate of ventilation should be selected considering the trade-off.

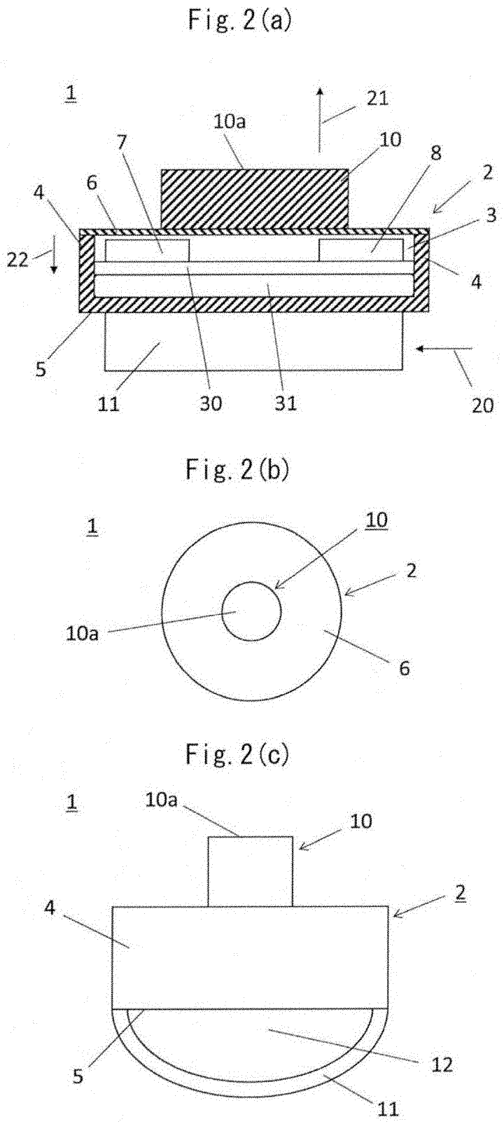

FIGS. 2 to 5 illustrate other embodiments of the measurement device 1 described using FIG. 1. The parts common to the structure in FIG. 1 are designated by the same reference numerals and the description thereof will be omitted.

The measurement device 1 illustrated in FIGS. 2(a)-2(c) is provided with a holding means 11 on the outside of the bottom face 5 of the measurement portion 2 (lower side in FIG. 2c).

The illustrated holding means 11 includes a cylindrical hollow portion 12 into which the tip of a finger of a human hand is inserted, as illustrated in FIG. 2(c). The person using the measurement device 1 can hold the measurement device 1 with his/her finger inserted into the cylindrical hollow portion 12, bring the contact surface 10a of the pressing portion 10 into contact with the eyelid, and press the measurement device 1 as indicated by arrow 21, so that the measurement device 1 becomes easy to handle.

Examples of the form of the holding means 11 that has the cylindrical hollow portion into which the tip of a finger of a human hand is inserted include a structure like a finger cot for counting the number of sheets of paper attached to the tip of a finger and a structure that is tied with a belt to a finger. The part of the finger cot-shaped holding means 11 where the finger is inserted can be made of an elastic material. The material of the belt may be elastic.

It is possible to achieve stable holding by forming the part on the cylindrical hollow portion of the holding means 11 where the finger comes into contact in a concave shape that fits the shape of the finger.

Note that, in FIG. 2(a), the wall face 6 is thinner than the bottom face 5 and the cylindrical wall face 4, considering the flexural bending toward the internal hollow portion 3 as indicated by arrow 22.

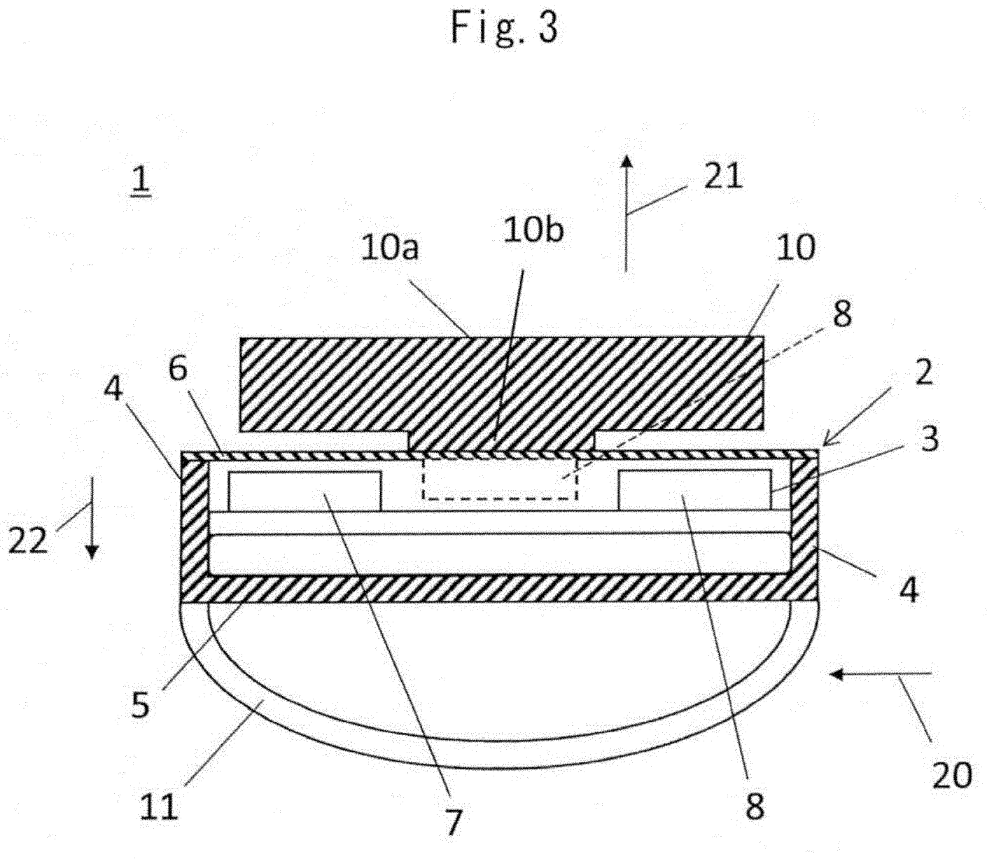

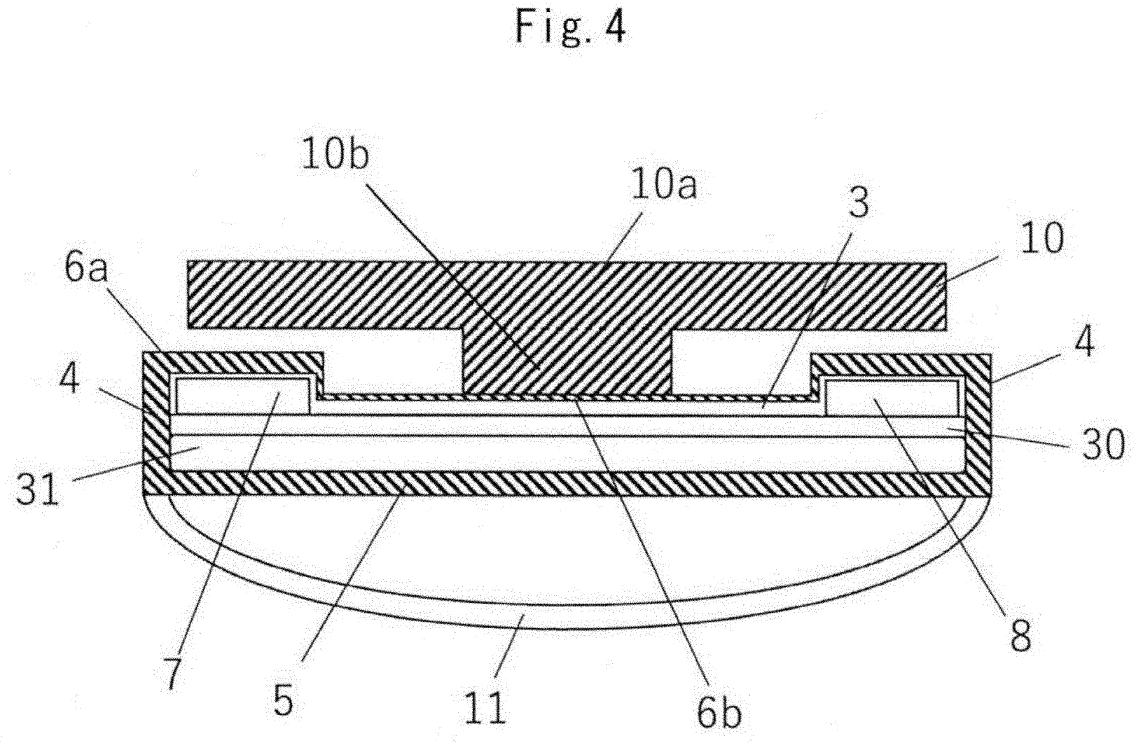

In FIGS. 3 and 4, similar to FIG. 1, the pressing portion 10 is erected on the wall face 6 via the pressing portion rear 10b that has a small diameter.

The smaller the diameter of the pressing portion rear 10b, when the contact surface 10a of the pressing portion 10 is brought into contact with the eyelid and the measurement device 1 is pressed in the direction indicated by arrow 21, the more the wall face 6 is likely to be bent in the direction of arrow 22.

In FIG. 3, similar to FIGS. 1 and 2, the second sensing means 8 is mounted on the substrate 30 on the battery 31. Alternatively, the measurement portion 2 may have a structure in which the second sensing means 8 is placed on the inner surface of the wall face 6 at the position where the pressing portion 10 is erected on the wall face 6, as illustrated by the broken line.

Similar to the embodiments illustrated in FIGS. 1 and 2, the first sensing means 7 placed on the substrate 30 closer to the bottom face 5 is a sensing means that continuously senses the internal pressure of the internal hollow portion 3 that changes according to the wall face 6 bending toward the internal hollow portion 3. The second sensing means 8 is a sensing means that continuously senses the distance of movement D of the pressing portion 10 toward the eyelid when the contact surface 10a is brought into contact with the eyeball or eyelid and the pressing portion 10 is pressed toward the eyeball or eyelid.

In the embodiment illustrated in FIG. 1, the second sensing means 8 senses the distance of movement of the entire measurement portion 2.

The magnitude of the flexural bending of the wall face 6 in the direction of the internal hollow portion 3, indicated by arrow 22, due to the repulsive force F can be extremely small.

In the embodiment illustrated in FIG. 1, in which the second sensing means 8 senses the distance of movement of the entire measurement portion 2, the distance of movement of the pressing portion 10 toward the eyelid may be calculated considering the flexural bending of the wall face 6 (the central portion 6b) in the direction of the internal hollow portion 3 indicated by arrow 22, although the flexural bending is extremely small.

A structure in which a second sensing means 8 constituted by an accelerometer is placed on the inner surface of the wall face 6 at the position where the pressing portion 10 is erected on the wall face 6 is advantageous in sensing the distance of movement of the pressing portion 10 toward the eyelid.

Then, the second sensing means 8 may be mounted on a flexible printed circuit (FPC) board.

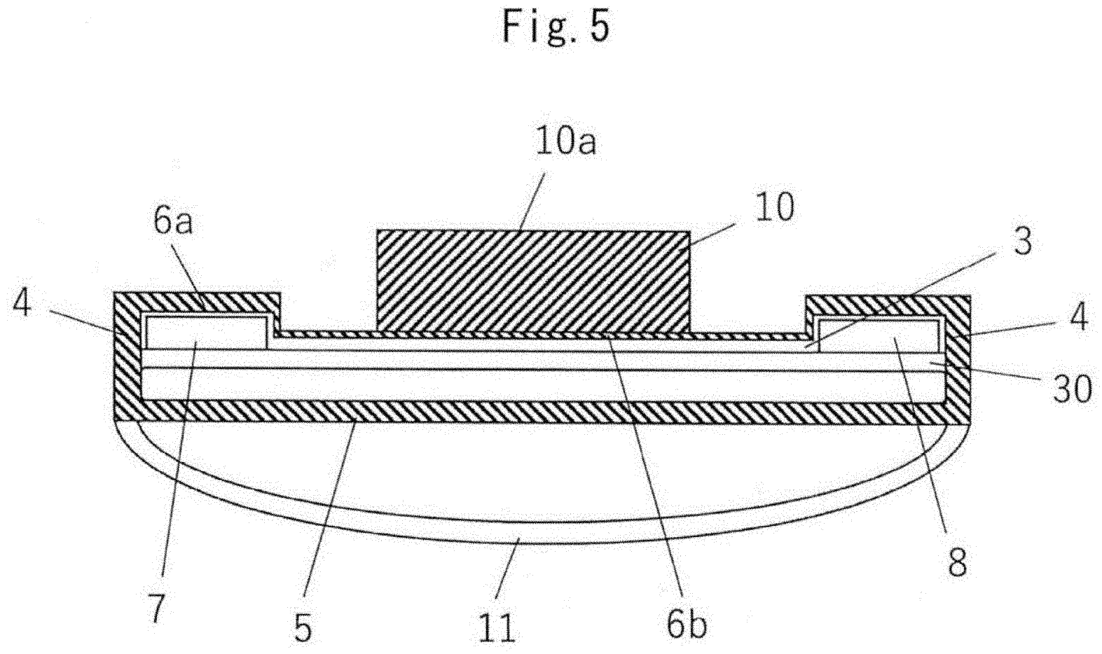

The structure illustrated in FIGS. 4 and 5 can suppress the height of the measurement device 1, similar to the structure illustrated in FIG. 1.

Also, in FIGS. 3, 4, and 5, the wall face 6 is thinner than the bottom face 5 and the cylindrical wall face 4, considering the flexural bending toward the internal hollow portion 3, indicated by arrow 22.

In addition, as illustrated in FIGS. 4 and 5, when the wall face 6 has a stepped structure formed by the central portion and the outer peripheral portion in the radial direction as in the embodiment illustrated in FIG. 1, the thickness of each portion can be adjusted considering the flexural bending as described in the embodiment illustrated in FIG. 1.

The embodiments illustrated in FIGS. 1 to 5 describe the structure where the pressing portion rear 10b of the pressing portion 10 is erected on the outside of one wall face of the measurement portion 2. Instead, as later described in Example 5, the pressing portion 10 and the measurement portion 2 can be separate in the embodiments illustrated in FIGS. 1 to 5.

Using this example, a synergy brought by the miniaturization of the measurement device 1 is summarized as follows. The smaller the measurement device 1 is, the better operability is when pressing, and the more desirable the situation is for calculating the distance by double-integrating acceleration, as described above. Therefore, the distance of movement D can be measured using the accelerometer. For that accelerometer, a small size is available, which contributes to further miniaturization of the measurement device 1. In addition, the good operability suppresses the rotation during the press, hence the direction of gravity due to the rotation has little influence on the accelerometer output. Further, since the good operability contributes to non-invasiveness, the measurement device 1 is suitable for applications where the subject measures intraocular pressure by himself/herself. The smaller the measurement device 1 and the internal hollow portion 3 are, the larger the pressure change appears even though the amount of bending of the wall face 6 is small, and the S/N ratio of the pressure sensor output improves. If the pressure sensor has low noise, the repulsive force can be sensed with a smaller amount of bending, and also the linear region in FIG. 12 can be used. For the pressure sensor, a small size is available, which contributes to further miniaturization of the measurement device 1.

### Example 2

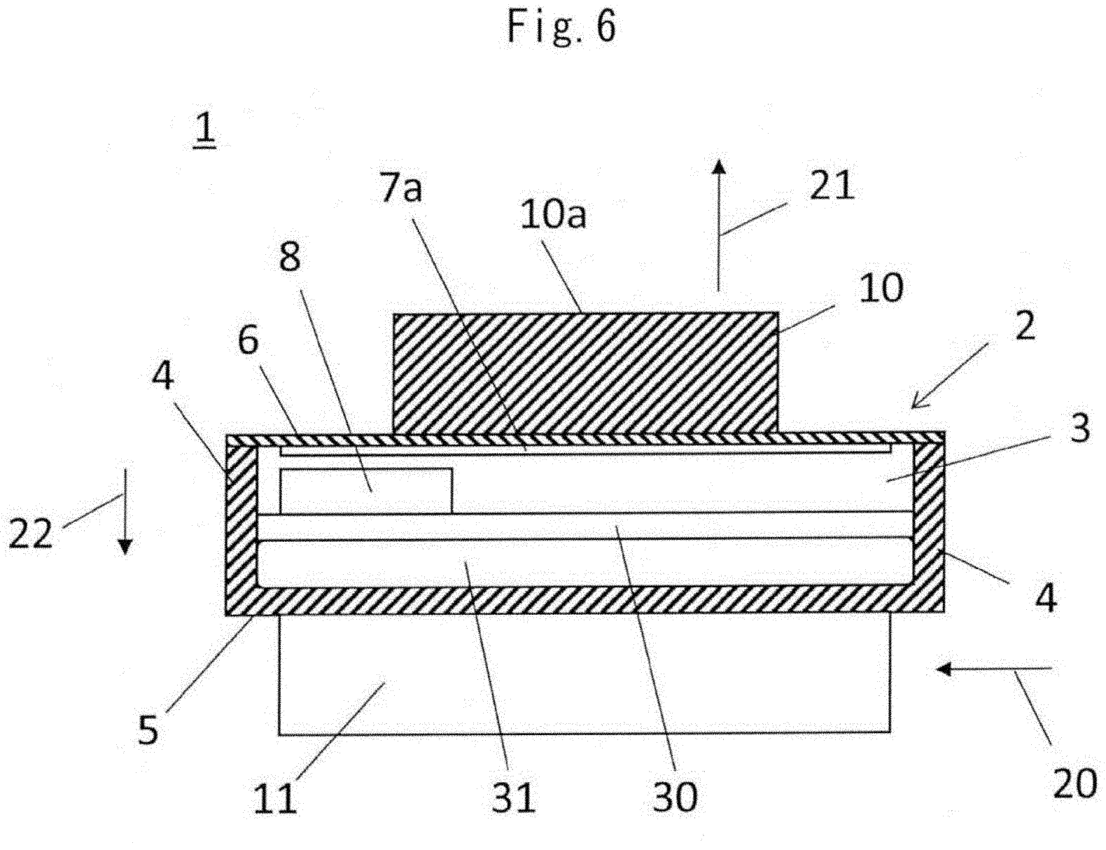

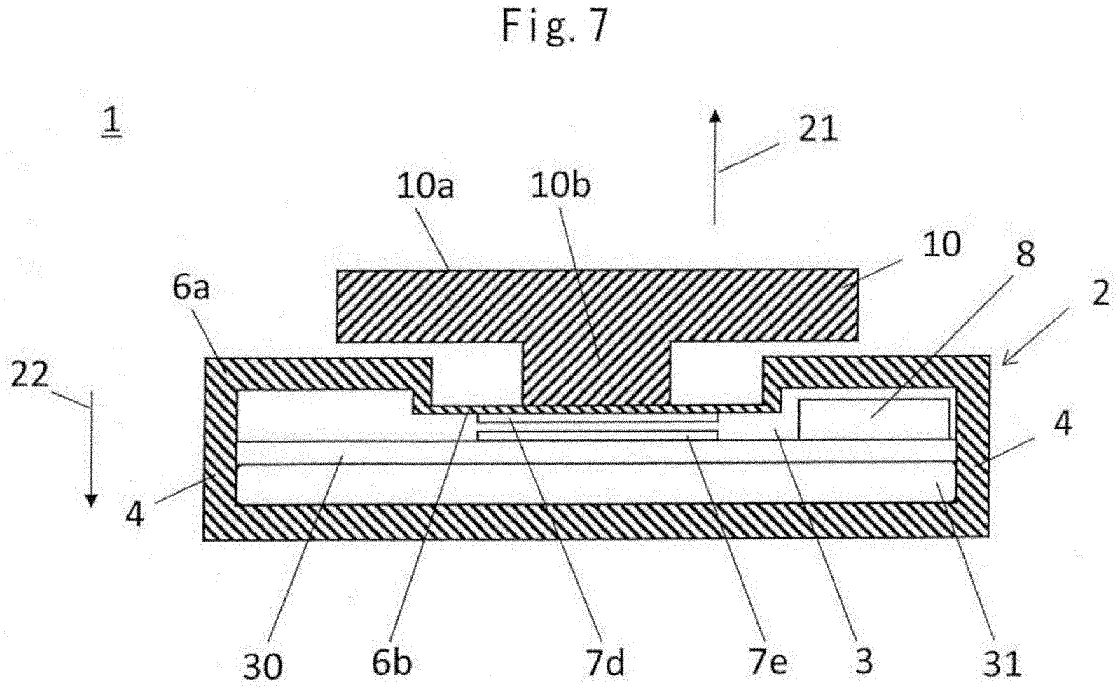

FIGS. 6 and 7 illustrate another example of the measurement device 1 illustrated in FIGS. 1 and 2. The parts common to the embodiments in FIGS. 1 and 2 are designated by the common reference numerals and the description thereof will be omitted.

The measurement portion 2 of the measurement device 1 illustrated in FIGS. 6 and 7 is not a sealed structure but a hollow structure that has simply the internal hollow portion 3 inside.

In FIG. 6, the pressing portion 10 is erected directly on the wall face 6 without the pressing portion rear 10b as opposed to FIG. 1. In this structure, the pressing portion rear of the pressing portion 10 is erected on the outside of one wall face 6 of the hollow structure.

In the embodiment illustrated in FIG. 6, the first sensing means 7a is placed on the inner surface of the wall face 6.

The first sensing means 7a may be placed on the outer surface of the wall face 6.

With the first sensing means 7a, the repulsive force F described above is continuously sensed by continuously sensing the amount of bending of the wall face 6 toward the internal hollow portion 3 by bringing the contact surface 10a into contact with the eyeball or eyelid and pressing the pressing portion 10 toward the eyeball or eyelid.

For example, the first sensing means 7a placed on the wall face 6 continuously senses the amount of bending by a variable resistor whose resistance changes according to the amount of bending of the wall face 6. An example of a variable resistor is a strain gauge to continuously sense the value of the resistance.

FIG. 7 illustrates a case where a different type of first sensing means is adopted compared to the embodiment illustrated in FIG. 6.

Here, a sensing means adopted continuously senses change in distance between the inner surface of the wall face 6 facing the internal hollow portion 3 and the inner surface of the bottom face 5 at the opposite side of the internal hollow portion 3 from the inner surface of the wall face 6. The change in distance is caused by the wall face 6 bending toward the internal hollow portion 3. For example, such a sensing means is an element whose electrical characteristics change according to the distance between the inner surfaces of the wall face 6 and the bottom face 5.

In FIG. 7, an electrode 7d is placed on the inner surface of the wall face 6, and an electrode 7e is placed on the inner surface of the bottom face 5 that faces the inner surface of the wall face 6. By continuously sensing that the capacitance between both electrodes changes as the wall face 6 bends toward the internal hollow portion 3, it is possible to continuously sense the repulsive force F.

Also in this example, as illustrated in FIG. 12, the relationship between the output of the strain gauge, the amount of change in the capacitance, or the like according to the amount of bending of the wall face 6 toward the internal hollow portion 3, which is sensed by the first sensing means, and the repulsive force F is known in advance. This relationship depends on the shape and member of the hollow structure. Accordingly, based on the predetermined relationships and information known in advance, the information processing described above using FIGS. 10 and 11 is subsequently performed by a predetermined information processing means (for example, a microcontroller), and intraocular pressure can be calculated by the measurement device. Algorithms, conversion formulas, parameters, which are required for information processing to obtain the repulsive force F from the output of the strain gauge sensed by the first sensing means according to the amount of bending of the wall face 6 toward the internal hollow portion 3 or from the relationship between the amount of change in the capacitance and the repulsive force F, and the information processing described above using FIGS. 10, 11, and 12 are stored in a storage unit (for example, a built-in non-volatile memory in the microcontroller) and are referred to.

In FIGS. 6 and 7, the thickness of the wall faces 6 and 6b is thinner than that of the bottom face 5 and the cylindrical wall face 4 considering the flexural bending toward the internal hollow portion 3, indicated by arrow 22.

Note that, in this example, the amount of bending of the wall face 6 and the wall face 6b is can be so small as to be negligible, as compared with the distance of movement of the pressing portion 10 toward the eyeball or eyelid. Therefore, the second sensing means 8 can be placed on the substrate 30 instead of on the pressing portion 10.

Since other operations and functions are the same as those described in Example 1, the description thereof will be omitted.

Note that, in the embodiments illustrated in FIGS. 6 and 7, the pressing portion 10 is fixed to the measurement portion 2, but instead, it is possible to configure the pressing portion 10 and the measurement portion 2 as separate bodies, as later described in Example 5, in the embodiments illustrated in FIGS. 6 and 7.

### Example 3

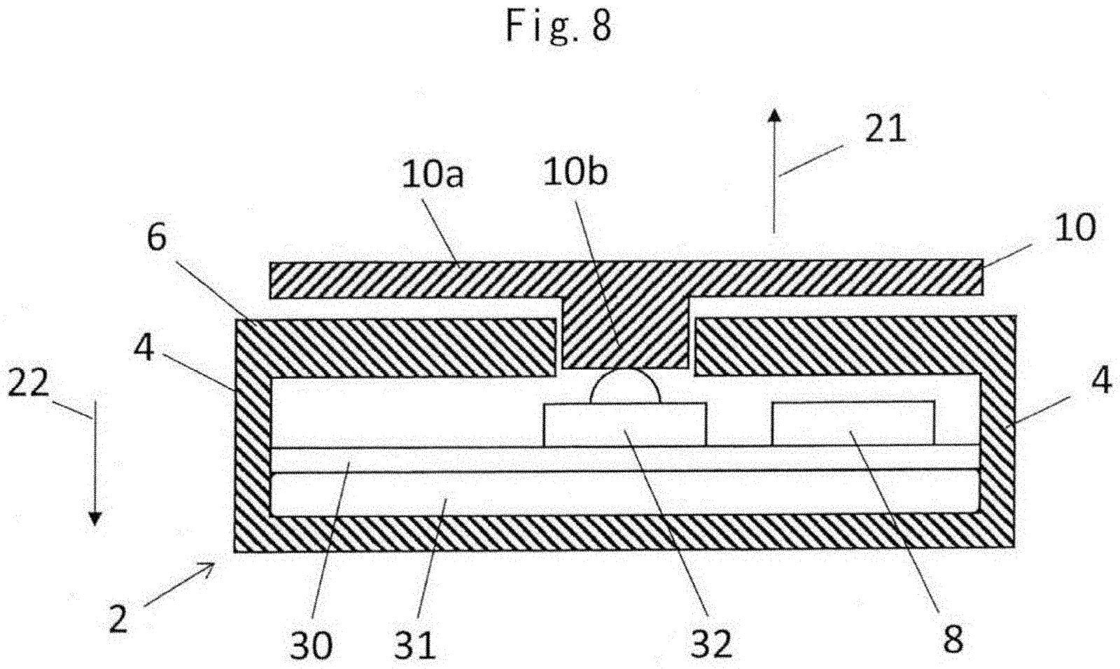

FIG. 8 illustrates another example of the measurement device of the present invention used as an intraocular pressure measurement device.

The parts common to the structure in FIG. 1 are designated by the same reference numerals and the description thereof will be omitted.

In the intraocular pressure measurement device illustrated in FIG. 8, a hole is provided in the center of the wall face 6. The pressing portion rear 10b is slidably inserted in this hole. By this structure, the pressing portion rear 10a of the pressing portion 10 is slidably supported by the measurement portion 2.

The measurement portion 2 is structured such that the first sensing means 32 formed on the substrate 30 comes into contact with the rear of the pressing portion 10 inside the measurement portion 2.

As the first sensing means 32, as long as the repulsive force F in the direction of arrow 22 from the eyeball when the contact surface 10a is brought into contact with the eyelid and the pressing portion 10 is pressed in the direction, indicated by arrow 21, can be continuously sensed, various types such as a force sensor, a touch-sensitive pressure sensor, a load cell, a capacitance sensor, and the like can be adopted.

Also, in this example, as illustrated in FIG. 12, the relationship between the output from the various sensors described above constituting the first sensing means 32 and the repulsive force F is known in advance.

In Example 1, the repulsive force F from the eyeball was calculated from the change in the internal pressure of the internal hollow portion 3 sensed by the first sensing means 7 based on the relationship illustrated in FIG. 12. In Example 2, the repulsive force F from the eyeball is calculated from the output of the strain gauge or the change in the capacitance according to the amount of bending of the wall face 6 sensed by the first sensing means, based on the relationship illustrated in FIG. 12. In Example 3, the repulsive force F from the eyeball is calculated from the output sensed by the first sensing means 32 based on the relationship illustrated in FIG. 12.

That is, also in this example, based on the relationships and information illustrated in FIG. 12, the information processing described above using FIGS. 10 and 11 is subsequently performed by a predetermined information processing means (for example, a microcontroller), and the intraocular pressure can be calculated by the measurement device. Algorithms, conversion formulas, and parameters required for the information processing described above using FIGS. 10, 11, and 12 are stored in a storage unit (for example, a built-in non-volatile memory in the microcontroller) and referred to.

In this example, it is not required to measure the amount of bending of the wall face 6 toward the internal hollow portion by bringing the contact surface 10a into contact with the eyeball or eyelid and pressing the pressing portion 10 toward the eyeball or eyelid. Therefore, it is not required to select the member of the wall face 6 considering the flexural bending as opposed to Examples 1 and 2.

When the pressing portion 10 is pressed in the direction of the eyeball or eyelid, the distance that the pressing portion 10 moves downward in FIG. 8 due to the repulsive force F from the eyeball is negligible compared with the distance of movement of the pressing portion 10 in the direction of the eyeball or eyelid. Therefore, the second sensing means 8 can be placed on the substrate 30 instead of on the pressing portion 10.

Since other operations and functions are the same as those described in Example 1, the description thereof will be omitted.

### Example 4

FIGS. 13(a) and 13(b) illustrate another example of the measurement device of the present invention used as an intraocular pressure measurement device. The example illustrated in FIGS. 13(a) and 13(b) are embodiments of a structure where the pressing portion is movably supported by the measurement portion so that the pressing portion can move in the direction from the tip to the rear of the pressing portion.

The parts common to the structure in FIGS. 2(a)-2(c) are designated by the same reference numerals and the description thereof will be omitted. In the intraocular pressure measurement device illustrated in FIGS. 13(a) and 13(b), the wall face 6 does not bend.

The embodiments illustrated in FIGS. 13(a) and 13(b) are different from FIGS. 1 to 7 in that it is not the bending of the wall face 6 that changes the volume of the internal hollow portion 3, and in that one wall face 6 constituting the measurement portion 2 plays the role of the pressing portion 10 in Examples 1 to 3, as will be described later.

In the embodiments illustrated in FIGS. 13(a) and 13(b), one wall face 6 that constitutes the measurement portion 2 is constituted by a member that is rigid enough not to bend when a pressing force is applied in the direction of arrow 22.

In the embodiments illustrated in FIGS. 13(a) and 13(b), a cylindrical support portion 4a is provided inside a cylindrical wall face 4 of the measurement portion 2, and the O-ring 33 is supported by the cylindrical support portion 4a. The wall face 6 placed on the upper side of an O-ring 33 is movable in the vertical direction in the cylindrical wall face 4 in the drawing. The measurement portion 2 is structured such that the internal hollow portion 3 is sealed by the O-ring 33.

In FIG. 13(a), when the measurement device 1 is pressed in the direction of the measured object (eyeball or eyelid) as indicated by arrow 21, the wall face 6 is also pressed in the direction of the measured object (eyeball or eyelid) as indicated by arrow 21. And then, the wall face 6 moves (goes down) in the direction of the internal hollow portion 3 as indicated by arrow 22. The mechanism is similar to above-described Example 1 in that the repulsive force from the measured object is continuously sensed by the first sensing means 7 according to the change in the volume and internal pressure of the internal hollow portion 3.

In FIG. 13(b), the mechanism is similar to above-described Example 2 in that the distance of movement of the wall face 6, which corresponds to the pressing portion member, in the direction of the internal hollow portion 3 due to the movement (sink) toward the internal hollow portion 3 is continuously sensed by the first sensing means 7.

Since the sensing operation by the second sensing means 8 in FIGS. 13(a) and 13(b) is similar to Examples 1 to 3, the explanation of which will be omitted.

In the above description, the internal hollow portion 3 is sealed by an elastic sealing structure, that is, by using the O-ring 33 as a sealing material that serves as the sealing structure and also as an elastic body. Instead of the O-ring, an elastic body such as a gasket and the like or springs that are supported by the cylindrical support portion 4a and placed along the inner circumference of the cylindrical wall face 4 can also be adopted. When adopting springs, sealing of the side face can be realized with an elastic material and/or structure, or with a bellows structure that expands and contracts as seen in a speaker cone or a musical instrument accordion. Options for the sealing material in this example are those that allow for change in the volume of the internal hollow portion 3 and also allow for sealing of the internal hollow portion 3, when the wall face 6 moves (sinks) toward the internal hollow portion 3 as indicated by arrow 22.

The embodiment illustrated in FIG. 13(b) is different from FIG. 13(a) in that the electrodes 7d and 7e constitute the first sensing means as in FIG. 7. In FIG. 13(b), the electrode 7e is supported by a support base 7f. By adjusting the thickness of the support base 7f, the distance between the electrode 7d and the electrode 7e can be narrowed, and a large capacitance can be configured.

In the embodiments of FIGS. 13(a) and 13(b), the pressing portion 10 in which the pressing portion rear 10b is supported by the measurement portion 2 as described in Examples 1 to 3 is not illustrated. This is because one wall face 6 that constitutes the measurement portion 2 in this example plays the role of the pressing portion 10 that is described in Examples 1 to 3.

In case of the embodiments illustrated in FIGS. 13(a) and 13(b), the size of the wall face 6 that corresponds to the pressing portion in the left-right direction in the drawing can be set to around 10 mm. It is possible to measure intraocular pressure by putting the lower side of the measurement portion 2 on the pad of a human finger.

Since other operations and functions are the same as those described in Examples 1 to 3, the description thereof will be omitted.

### Example 5

In FIG. 14, as in Example 4 (FIGS. 13(a) and 13(b)), one wall face 6 that constitutes the measurement portion 2 is constituted by a member that is rigid enough not to bend when a pressing force is applied in the direction of arrow 22. The pressing portion 10 is placed on the one wall face 6.

Also, in this embodiment, the pressing portion 10 and the measurement portion 2 are separate.

In FIG. 14, the measurement portion indicated by reference numeral 2 is a measurement portion that has the functions of the first sensing means 7 and the second sensing means 8 described above. In addition, the pressing portion rear 10b of the pressing portion 10 is configured to be able to be attached to/removed from the outside of the wall face 6 of the measurement portion 2 by an attachment/detachment means (not illustrated in the drawings).

Other parts common to the structures in FIGS. 2 and 13(a)/13(b) are designated by the same reference numerals and the description thereof will be omitted.

As the structure in which the pressing portion 10 and the measurement portion 2 are separate and the pressing portion rear 10b of the pressing portion 10 can be attached to/removed from the measurement portion 2, a structure in which the pressing portion rear 10b is mounted on the outside of the wall face 6 can be adopted as a form in which the pressing portion rear 10b of the pressing portion 10 is supported by the measurement portion 2.

For example, various things such as an adhesive tape, a suction sheet, a magnetic force between the measurement portion 2 and the pressing portion 10, one or more suction cups provided on the bottom face of the pressing portion 10, and the like can be adopted. To mount the pressing portion 10 in the center of the wall face 6, a mark of the mounting position can be put on the wall face 6 in advance.

As the measurement portion 2 that has the functions of the first sensing means 7 and the second sensing means 8, an electronic/electrical device that has such functions can be exemplified. As the electronic/electronic device in this case, for example, a smartwatch and a smartphone equipped with a pressure sensor and an accelerometer can be exemplified. Then, the wall face 6 is the display of the electronic/electrical device, which is advantageous because the mounting position can be displayed. The first sensing means can be constituted not only by the pressure sensor but also by any of the various components exemplified in Examples 1 to 3.

In the embodiment of FIG. 14, the size in the left-right direction can be set to several cm, which is the size of a smartwatch, and the size of the pressing portion 10 whose tip is intended to be in contact with the eyeball or eyelid can be set to approximately 10 mm in diameter.

Since other operations and functions are the same as those described in Examples 1 to 4, the description thereof will be omitted.

The pressing portion 10 and the measurement portion 2 can be separate in Examples 1 and 2 as well. That is, the configuration described in Example 1 where the volume of the internal hollow portion 3 is changed by bending of the wall face 6 (using the change in the internal pressure of the internal hollow portion 3) and the configuration described in Example 2 (where the amount of bending is reflected to the change in capacitance and the like) can be applied to the intraocular pressure measurement and the like by making the pressing portion 10 attachable to/detachable from the display of the smartwatch/smartphone as described in this Example 5.

The examples and the embodiments with reference to the accompanying drawings of the present invention have been described above, but the present invention is not limited to the example forms and examples described above and can be changed in various ways within the technical scope grasped from the description of the scope of claims.

1.-17. (canceled)

18. A measurement device for measuring internal pressure or rigidity of a measured object, the measurement device comprising:
a pressing portion; and
a measurement portion, wherein
the pressing portion has, at a tip, a contact surface that comes into contact with a surface of the measured object, and
the measurement portion includes:
a first sensing means configured to, when the contact surface is brought into contact with the surface of the measured object and the pressing portion is pressed toward the measured object, continuously sense repulsive force from the measured object; and
a second sensing means configured to continuously sense any one of:
speed of movement at which the pressing portion moves in a direction of the measured object;
acceleration of the pressing portion in a movement in the direction of the measured object; or
distance of movement of the pressing portion in the direction of the measured object,
the measurement portion is composed of a hollow structure having an internal hollow portion inside,
a pressing portion rear, which is the opposite side of the pressing portion from the tip, is supported on an outside of one wall face of the hollow structure, and
the first sensing means is configured to:
sense the repulsive force continuously by sensing continuously an amount of bending of the one wall face toward the internal hollow portion by bringing the contact surface into contact with the surface of the measured object and pressing the pressing portion toward the measured object; or
sense the repulsive force continuously by sensing continuously a change in pressure of the internal hollow portion generated by bending the one wall face toward the internal hollow portion by bringing the contact surface into contact with the surface of the measured object and pressing the pressing portion toward the measured object.

19. The measurement device according to claim 18, wherein
the hollow structure is any one of:
a sealed structure, a hollow structure having a hole for communicating between the internal hollow portion and an external space, or a hollow structure in which a breathable film is provided between the internal hollow portion and the external space.

20. The measurement device according to claim 18, wherein
the pressing portion and the measurement portion are separate, and the pressing portion rear is attachable to and removable from the measurement portion.

21. A measurement device for measuring internal pressure or rigidity of a measured object, the measurement device comprising:
a pressing portion; and
a measurement portion, wherein
the pressing portion has, at a tip, a contact surface that comes into contact with a surface of the measured object, and
the measurement portion includes:
a first sensing means configured to, when the contact surface is brought into contact with the surface of the measured object and the pressing portion is pressed toward the measured object, continuously sense repulsive force from the measured object; and
a second sensing means configured to continuously sense any one of:
speed of movement at which the pressing portion moves in a direction of the measured object;
acceleration of the pressing portion in a movement in the direction of the measured object; or
distance of movement of the pressing portion in the direction of the measured object,
the measurement portion is composed of a hollow structure having an internal hollow portion inside,
the pressing portion is movably supported by the hollow structure to be movable toward the internal hollow portion, and
the first sensing means is configured to sense the repulsive force continuously by sensing continuously an amount of movement of the pressing portion toward the internal hollow portion by bringing the contact surface into contact with the surface of the measured object and pressing the pressing portion toward the measured object.

22. The measurement device according to claim 21, wherein
the first sensing means continuously senses the repulsive force by bringing a pressing portion rear, which is the opposite side of the pressing portion from the tip, into contact with the first sensing means in a direction toward the internal hollow portion.

23. A measurement device for measuring internal pressure or rigidity of a measured object, the measurement device comprising:
a pressing portion; and
a measurement portion, wherein
the pressing portion has, at a tip, a contact surface that comes into contact with a surface of the measured object, and
the measurement portion includes:
a first sensing means configured to, when the contact surface is brought into contact with the surface of the measured object and the pressing portion is pressed toward the measured object, continuously sense repulsive force from the measured object; and
a second sensing means configured to continuously sense any one of:
speed of movement at which the pressing portion moves in a direction of the measured object;
acceleration of the pressing portion in a movement in the direction of the measured object; or
distance of movement of the pressing portion in the direction of the measured object,
the measurement portion is composed of a hollow structure having an internal hollow portion inside,
a pressing portion rear, which is the opposite side of the pressing portion from the tip, is supported on an outside of one wall face of the hollow structure, and
the first sensing means is configured to:
sense the repulsive force continuously by sensing continuously a change in pressure of the internal hollow portion generated by moving the pressing portion toward the internal hollow portion by bringing the contact surface into contact with the surface of the measured object and pressing the pressing portion toward the measured object;
sense the repulsive force continuously by sensing continuously an amount of movement of the one wall face toward the internal hollow portion by bringing the contact surface into contact with the surface of the measured object and pressing the pressing portion toward the measured object; or
sense the repulsive force continuously by sensing continuously a change in the pressure of the internal hollow portion generated by moving the one wall face toward the internal hollow portion by bringing the contact surface into contact with the surface of the measured object and pressing the pressing portion toward the measured object.

24. The measurement device according to claim 23, wherein
the one wall face is movably supported by the hollow structure to be movable toward the internal hollow portion.

25. The measurement device according to claim 23, wherein
the pressing portion and the measurement portion are separate, and the pressing portion rear is attachable to and removable from the measurement portion.

26. A measurement device for measuring internal pressure or rigidity of a measured object, the measurement device comprising a measurement portion, wherein
the measurement portion is composed of a hollow structure having an internal hollow portion inside, one wall face among a plurality of wall faces forming the hollow structure is movably supported by the hollow structure to be movable toward inside of the hollow portion, an inner surface of the one wall face faces the internal hollow portion and an outer surface of the one wall face forms a contact surface that comes into contact with a surface of the measured object,
the measurement portion includes:
a first sensing means configured to, when the contact surface formed by the outer surface is brought into contact with the surface of the measured object and the one wall face is pressed toward the measured object, continuously sense repulsive force from the measured object; and
a second sensing means configured to continuously sense any one of:
speed of movement at which the wall face moves in a direction of the measured object;
acceleration of the wall face in a movement in the direction of the measured object; or
distance of movement of the wall face in the direction of the measured object,
the first sensing means is configured to:
sense the repulsive force continuously by sensing continuously a change in pressure of the internal hollow portion generated by moving the one wall face toward the internal hollow portion by bringing the contact surface into contact with the surface of the measured object and pressing the one wall face toward the measured object, or
sense the repulsive force continuously by sensing continuously an amount of movement of the one wall face toward the internal hollow portion by bringing the contact surface into contact with the surface of the measured object and pressing the one wall face toward the measured object.

27. The measurement device according to claim 23, wherein
the hollow structure is any one of:
a sealed structure, a hollow structure having a hole for communicating between the internal hollow portion and an external space, or a hollow structure in which a breathable film is provided between the internal hollow portion and the external space.

28. The measurement device according to claim 23, wherein
the hollow structure is a sealed structure, and
a sealing material that seals the hollow structure has elasticity, and the sealing material enables a change in volume of the internal hollow portion caused by a movement of the pressing portion toward the internal hollow portion.

29. The measurement device according to claim 26, wherein
the hollow structure is a sealed structure, and
a sealing material that seals the hollow structure has elasticity, and the sealing material enables a change in volume of the internal hollow portion caused by a movement of the one wall face toward the internal hollow portion.

30. The measurement device according to claim 18, wherein
the measurement portion is an electronic or electrical device including the first sensing means and the second sensing means.

31. The measurement device according to claim 18, further comprising a third sensing means configured to continuously sense a rotational movement that occurs to the pressing portion when the contact surface is brought into contact with the surface of the measured object and the pressing portion is pressed toward the measured object.

32. The measurement device according to claim 26, further comprising a third sensing means configured to continuously sense a rotational movement that occurs to the one wall face when the contact surface is brought into contact with the surface of the measured object and the one wall face is pressed toward the measured object.

33. The measurement device according to claim 18, further comprising a third sensing means for continuously sensing a change in a direction of gravity with respect to the second sensing means.

34. A measurement device for measuring internal pressure or rigidity of a measured object, the measurement device comprising:
a pressing portion; and
a measurement portion, wherein
the pressing portion has, at a tip, a contact surface that comes into contact with a surface of the measured object, and
the measurement portion includes:
a first sensing means configured to, when the contact surface is brought into contact with the surface of the measured object and the pressing portion is pressed toward the measured object, continuously sense repulsive force from the measured object;
a second sensing means configured to continuously sense acceleration of the pressing portion in a movement in a direction of the measured object; and
a third sensing means configured to continuously sense a rotational movement that occurs to the pressing portion when the contact surface is brought into contact with the surface of the measured object and the pressing portion is pressed toward the measured object.

35. A measurement device for measuring internal pressure or rigidity of a measured object, the measurement device comprising:
a pressing portion; and
a measurement portion, wherein
the pressing portion has, at a tip, a contact surface that comes into contact with a surface of the measured object, and
the measurement portion includes:
a first sensing means configured to, when the contact surface is brought into contact with the surface of the measured object and the pressing portion is pressed toward the measured object, continuously sense repulsive force from the measured object;
a second sensing means configured to continuously sense acceleration of the pressing portion in a movement in a direction of the measured object; and
a third sensing means configured to continuously sense a change in a direction of gravity with respect to the second sensing means.

36. A measurement device for measuring internal pressure or rigidity of a measured object, the measurement device comprising a measurement portion, wherein
the measurement portion is composed of a hollow structure having an internal hollow portion inside, one wall face among a plurality of wall faces forming the hollow structure is movably supported by the hollow structure to be movable toward inside of the hollow portion, an inner surface of the one wall face faces the internal hollow portion and an outer surface of the one wall face forms a contact surface that comes into contact with a surface of the measured object, and
the measurement portion includes:
a first sensing means configured to, when the contact surface formed by the outer surface is brought into contact with the surface of the measured object and the one wall face is pressed toward the measured object, continuously sense repulsive force from the measured object;
a second sensing means configured to continuously sense acceleration of the one wall face in a movement in a direction of the measured object; and
a third sensing means configured to continuously sense a rotational movement that occurs with the one wall face when the contact surface is brought into contact with the surface of the measured object and the one wall face is pressed toward the measured object.

37. A measurement device for measuring internal pressure or rigidity of a measured object, the measurement device comprising a measurement portion, wherein
the measurement portion is composed of a hollow structure having an internal hollow portion inside, one wall face among a plurality of wall faces forming the hollow structure is movably supported by the hollow structure to be movable toward inside of the hollow portion, an inner surface of the one wall face faces the internal hollow portion and an outer surface of the one wall face forms a contact surface that comes into contact with a surface of the measured object, and
the measurement portion includes:
a first sensing means configured to, when the contact surface formed by the outer surface is brought into contact with the surface of the measured object and the one wall face is pressed toward the measured object, continuously sense repulsive force from the measured object;
a second sensing means configured to continuously sense acceleration of the one wall face in a movement in a direction of the measured object; and
a third sensing means configured to continuously sense a change in a direction of gravity with respect to the second sensing means.

38. The measurement device according to claim 18, wherein
the internal pressure of the measured object is intraocular pressure, and the contact surface is brought into contact with an eyeball or an eyelid.

39. The measurement device according to claim 38, wherein
force that brings the contact surface into contact with the eyeball or the eyelid and presses the pressing portion toward the eyeball or the eyelid is given by a pressing operation by a human hand.

40. The measurement device according to claim 26, wherein
the internal pressure of the measured object is intraocular pressure, and the contact surface is brought into contact with an eyeball or an eyelid.

41. The measurement device according to claim 40, wherein
force that brings the contact surface into contact with the eyeball or the eyelid and presses the one wall face toward the eyeball or the eyelid is given by a pressing operation by a human hand.
